(19) United States
(12) Patent Application Publication
(10) Pub. No.: US 2007/0037790 A1
(43) Pub. Date: Feb. 15, 2007

(54) Bicyclo-pyrazole derivatives active as kinase inhibitors, process for their preparation and pharmaceutical compositions comprising them

(76) Inventors: Francesca Abrate, Milan (IT); Daniele Fancelli, Milan (IT); Mario Varasi, Milan (IT); Manuela Villa, Como (IT)

Correspondence Address: SCULLY, SCOTT, MURPHY & PRESSER, 400 GARDEN CITY PLAZA, SUITE 300, GARDEN CITY, NY, 11530, US

(21) Appl. No.: 10/548,332

(22) PCT Filed: Mar. 2, 2004
(86) PCT No.: PCT/EP04/50237

§ 371 Date: Sep. 13, 2006

Related U.S. Application Data
(60) Provisional application No. 60/453,885, filed on Mar. 11, 2003.

(51) Int. Cl. A61K 31/55; A61K 31/4745

(52) U.S. Cl. 514/215; 514/303

(57) ABSTRACT

Compounds which are pyrrolo-pyrazole derivatives and pharmaceutically acceptable salts thereof, together with the process for their preparation and pharmaceutical compositions thereof are disclosed; these compounds or compositions are useful in the treatment of diseases caused by and/or associated with an altered protein kinase activity such as cancer, cell proliferative disorders, Alzheimer's disease, viral infections, auto-immune diseases and neurodegenerative disorders.

The present invention relates to bicyclo-pyrazole derivatives active as kinase inhibitors and, more in particular, it relates to pyrrolo-pyrazole derivatives, to a process for their preparation, to pharmaceutical compositions comprising them and to their use as therapeutic agents, particularly in the treatment of diseases linked to disregulated protein kinases.

The disregulation of protein kinases (PK) activity is the hallmark of numerous diseases. A large share of the oncogenes and proto-oncogenes involved in human cancers code for PKs. The enhanced activities of PKs are also implicated in many non-malignant diseases, such as benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis. PKs are also implicated in inflammatory conditions and in the multiplication of viruses and parasites. PKs may also play a major role in the pathogenesis and development of neurodegenerative disorders.

For a general reference to PKs malfunctioning or disregulation see, for instance, Current Opinion in Chemical Biology 1999, 3, 459-465.

It is an object of the invention to provide compounds which are useful in therapy as agents against a host of diseases caused by and/or associated to a disregulated protein kinase activity.

It is another object to provide compounds which are endowed with multiple protein kinase inhibiting activity.

The present inventors have now discovered that some pyrrolo-pyrazole derivatives are endowed with multiple protein kinase inhibiting activity and are thus useful in therapy in the treatment of diseases associated with disregulated protein kinases.

More specifically, the compounds of this invention are useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma

Due to the key role of PKs in the regulation of cellular proliferation, these pyrrolo-pyrazole derivatives are also useful in the treatment of a variety of cell proliferative disorders such as, for instance, benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

The compounds of the invention can be useful in the treatment of Alzheimer's disease, as suggested by the fact that cdk5 is involved in the phosphorylation of tau protein (J. Biochem., 117, 741-749, 1995).

The compounds of this invention, as modulators of apoptosis, may also be useful in the treatment of cancer, viral infections, prevention of AIDS development in HIV-infected individuals, autoimmune diseases and neurodegenerative disorders.

The compounds of this invention may be useful in inhibiting tumor angiogenesis and metastasis, as well as in the treatment of organ transplant rejection and host versus graft disease.

The compounds of the invention may also act as inhibitor of other protein kinases, e.g., protein kinase C in different isoforms, Met, PAK-4, PAK-5, ZC-1, STLK-2, DDR-2, Aurora 1, Aurora 2, Bub-1, PLK, Chk1, Chk2, HMER2, raf1, MEK1, MAPK, EGF-R, PDGF-R, FGF-R, IGF-R, PI3K, weel kinase, Src, Abl, Akt MAPK, ILK, MK-2, IKK-2, Cdc7, Nek, and thus be effective in the treatment of diseases associated with other protein kinases.

The compounds of the invention are also useful in the treatment and prevention of radiotherapy-induced or chemotherapy-induced alopecia.

Several heterocyclic compounds are known in the art as protein kinase inhibitors.

Among them are heterocyclic urea derivatives disclosed in WO 99/32455 as RAF kinase inhibitors, and aminopyrazole derivatives disclosed in WO 97/40019 as selective protein tyrosine kinase inhibitors. Amino-pyrimidine compounds are disclosed as p38 kinase inhibitors in WO 03/02544.

In addition, 2-carboxamido- and 2-ureido-pyrazole compounds have been disclosed as protein kinase inhibitors in the international patent applications WO 01/12189, WO 01/12188, WO 02/48114 and WO 02/70515, all in the name of the applicant itself.

Fused bicyclic compounds comprising pyrazole moieties and possessing kinase inhibitory activity have been also disclosed in WO 00/69846 and WO 02/12242, both in the name of the applicant itself.

The compounds object of the present invention fall within the scope of the general formula of the aforementioned WO 02/12242, herewith incorporated by reference, but are not specifically exemplified therein.

Accordingly, the present invention provides a method for treating diseases caused by and/or associated with an altered protein kinase activity, by administering to a mammal in need thereof an effective amount of a pyrazole represented by formula (I) 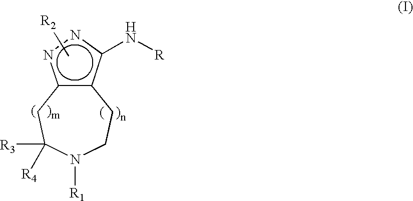
wherein
R is a hydrogen atom or a group selected from —COR′, —COOR′, —CONHR′, —C(═NH)NHR′, —SO₂R′ or —SO₂NR′R″;
R₁ is an optionally substituted 5 or 6 membered heterocyclic group with from 1 to 3 heteroatoms or heteroatomic groups selected from N, NR′, O or S, optionally benzocondensed;
R₂ is hydrogen or it is selected from the group consisting of R′, —COR′, —COOR′, —CONR′R″ or —S(O)_{q}R′;
R₃ and R₄ are both hydrogen atoms or methyl groups or, together with the carbon atom to which they are attached, form a cyclopropyl group;
R′ and R″ are, the same or different and independently in each of the above occasions, a hydrogen atom or an optionally substituted group selected from straight or branched C₁-C₆ alkyl C₃-C₆ cycloalkyl, aryl, aryl C₁-C₆ alkyl heterocyclyl or heterocyclyl C₁-C₆ alkyl;
m and n are 0 or 1, provided that they are not both 1;
q is 0 or an integer from 1 to 2;
and the pharmaceutically acceptable salts thereof.

In a preferred embodiment of the method described above, the disease caused by and/or associated with an altered protein kinase activity is selected from the group consisting of cancer, cell proliferative disorders, Alzheimer's disease, viral infections, auto-immune diseases and neurodegenerative disorders.

Specific types of cancer that may be treated include carcinoma, squamous cell carcinoma, hematopoietic tumors of myeloid or lymphoid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer and Kaposi's sarcoma.

In another preferred embodiment of the method described above, the cell proliferative disorder is selected from the group consisting of benign prostate hyperplasia, familial adenomatosis polyposis, neuro-fibromatosis, psoriasis, vascular smooth cell proliferation associated with atherosclerosis, pulmonary fibrosis, arthritis glomerulonephritis and post-surgical stenosis and restenosis.

In addition, the method object of the present invention, also provides tumor angiogenesis and metastasis inhibition.

The present invention further provides a pyrazole represented by formula (I) 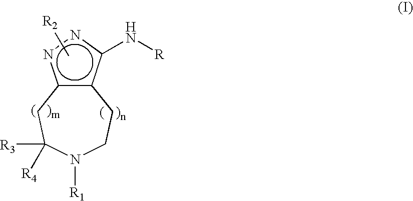
wherein
R is a hydrogen atom or a group selected from —COR′, —COOR′, —CONHR′, —C(═NH)NHR′, —SO₂R′ or —SO₂NR′R″;
R₁ is an optionally substituted 5 or 6 membered heterocyclic group with from 1 to 3 heteroatoms or heteroatomic groups selected from N, NR′, O or S, optionally benzocondensed;
R₂ is hydrogen or it is selected from the group consisting of R′, —COR′, —COOR′, —CONR′R″ or —S(O)_{q}R′;
R₃ and R₄ are both hydrogen atoms or methyl groups or, together with the carbon atom to which they are attached, form a cyclopropyl group;
R′ and R″ are, the same or different and independently in each of the above occasions, a hydrogen atom or an optionally substituted group selected from straight or branched C₁-C₆ alkyl, C₃-C₆ cycloalkyl, aryl, aryl C₁-C₆ alkyl, heterocyclyl or heterocyclyl C₁-C₆ alkyl;
m and n are 0 or 1, provided that they are not both 1;
q is 0 or an integer from 1 to 2;
and the pharmaceutically acceptable salts thereof.

The compounds of formula (I), object of the present invention, may have asymmetric carbon atoms and may therefore exist either as racemic admixtures or as individual optical isomers.

Accordingly, all the possible isomers and their admixtures and of both the metabolites and the pharmaceutically acceptable bio-precursors (otherwise referred to as pro-drugs) of the compounds of formula (I), as well as any therapeutic method of treatment comprising them, are also within the scope of the present invention.

In the present description, unless otherwise indicated, with the term straight or branched C₁-C₆ alkyl we intend a group such as, for instance, methyl, ethyl, n-propyl, isopropyl, n-butyl, isobutyl, sec-butyl tert-butyl n-pentyl, n-hexyl and the like.

With the term C₃-C₆ cycloalkyl we intend a group such as, for instance, cyclopropyl, cyclobutyl, cyclopentyl or cyclohexyl.

With the term aryl we intend a mono- or bi-either carbocyclic as well as heterocyclic group with from 1 to 2 ring moieties, either fused or linked to each other by single bonds, wherein at least one of the carbocyclic or heterocyclic rings is aromatic.

Non limiting examples of aryl groups are, for instance, phenyl, indanyl, biphenyl, α- or β-naphthyl, pyridyl, pyrazinyl, pyrimidinyl, pyridazinyl, 1,3,5-triazinyl, indolyl, imidazolyl, imidazopyridyl, 1,2-methylenedioxyphenyl, thiazolyl, isothiazolyl, pyrrolyl, pyrrolyl-phenyl, furyl, phenyl-furyl, benzotetrahydrofuranyl, oxazolyl, isoxazolyl, pyrazolyl, chromenyl, thienyl, benzothienyl, isoindolinyl, benzoimidazolyl, benzoxazolyl, benzothiazolyl, isoindolinyl-phenyl, quinolinyl, isoquinolinyl, quinoxalinyl pyrazinyl, benzofurazanyl, 1,2,3-triazolyl, 1-phenyl-1,2,3-triazolyl, and the like.

With the term 5 or 6 membered heterocycle, hence encompassing aromatic heterocyclic groups also referred to as aryl groups, we further intend a saturated or partially unsaturated 5 or 6 membered carbocycle wherein one or more carbon atoms are replaced by 1 to 3 heteroatoms or heteroatomic groups such as N, NR′, O or S, wherein R′ is as defined in the general formula.

Additional examples of 5 or 6 membered heterocyclic groups optionally benzocondensed or further substituted, besides those previously referred to as aryl groups, are 1,3-dioxolane, pyran, pyrrolidine, pyrroline, imidazolidine, pyrazolidine, pyrazoline, piperidine, piperazine, morpholine, tetrahydrofuran, and the like.

According to the above meanings provided to R₁, R′ and R″, any of the said groups may be further optionally substituted in any of the free positions by one or more groups, for instance 1 to 6 groups, selected from: halogen, nitro, oxo groups (═O), carboxy, cyano, alkyl, polyfluorinated alkyl, hydroxyalkyl alkenyl, alkynyl, cycloalkyl, aryl, heterocyclyl, amino groups and derivatives thereof such as, for instance, aminoalkyl, alkylaminoalkyl, dialkylaminoalkyl, alkylamino, dialkylamino, cycloalkylamino, arylamino, diarylamino, arylalkylamino, ureido, alkylureido or arylureido; carbonylamino groups and derivatives thereof such as, for instance, formylamino, alkylcarbonylamino, alkenylcarbonylamino, arylcarbonylamino, alkoxycarbonylamino; hydroxy groups and derivatives thereof such as, for instance, alkoxy, aryloxy, arylalkyloxy, heterocyclyloxy, alkylcarbonyloxy, arylcarbonyloxy, cycloalkenyloxy or alkylideneaminooxy; carbonyl groups and derivatives thereof such as, for instance, alkylcarbonyl, arylcarbonyl, alkoxycarbonyl, aryloxycarbonyl, cycloalkyloxycarbonyl, aminocarbonyl, alkylaminocarbonyl, dialkylaminocarbonyl; sulfurated derivatives such as, for instance, alkylthio, arylthio, alkylsufonyl, arylsulfonyl, alkylsulfinyl, arylsulfinyl, arylsulfonyloxy, aminosulfonyl, alkylaminosulfonyl or dialkylaminosulfonyl.

In their turn, whenever appropriate, each of the above groups may be further substituted by one or more of the aforementioned groups.

Among these latter groups and unless otherwise specified in the present description, with the term halogen atom we intend a fluorine, chlorine, bromine or iodine atom.

With the term polyfluorinated alkyl we intend a straight or branched C₁-C₆ alkyl group as above defined, wherein more than one hydrogen atom is replaced by fluorine atoms. Example of polyfluorinated alkyl groups are, for instance, trifluoromethyl 2,2,2-trifluoroethyl, 1,2-difluoroethyl, 1,1,1,3,3,3-hexafluoropropyl-2-yl and the like.

With the term alkenyl or alkynyl we intend a straight or branched unsaturated hydrocarbon chain having a double or triple bond, with from 2 to 6 carbon atoms such as, for instance, vinyl, ethynyl, 1-propenyl, alkyl, 1- or 2-propynyl, 1-, 2- or 3-butenyl, 1-, 2- or 3-butynyl, pentenyl, pentynyl, hexenyl, hexynyl and the like.

From all of the above, it is clear to the skilled person that any group which name has been identified as a composite name such as, for instance, cycloalkylalkyl, arylalkyl, heterocyclylalkyl, alkoxy, alkylthio, aryloxy, arylalkoxy, heterocyclyloxy, heterocyclylalkoxy, alkylcarbonyloxy and the lice, have to be intended as conventionally construed from the parts to which they derive.

As an example, the term heterocyclyl-alkyl stands for an alkyl group being further substituted by a heterocyclyl group, wherein alkyl and heterocyclyl are as above defined. Pharmaceutically acceptable salts of the compounds of formula (I) are the acid addition salts with inorganic or organic acids, e.g. nitric, hydrochloric, hydrobromic, sulfuric, perchloric, phosphoric, acetic, trifluoroacetic, propionic, glycolic, lactic, oxalic, malonic, malic, maleic, tartaric, citric, benzoic, cinnamic, mandelic, methanesulfonic, isethionic and salicylic acid, as well as the salts with inorganic or organic bases, e.g. alkali or alkaline-earth metals, especially sodium, potassium, calcium or magnesium hydroxides, carbonates or bicarbonates, acyclic or cyclic amines, preferably methylamine, ethylamine, diethylamine, triethylamine or piperidine.

When referring to the compounds of formula (I) of the invention, it is clear to the skilled person that the group R₂ may be linked to any one of the adjacent nitrogen atoms of the pyrazole ring, so as to give rise to two different tautomeric forms, as reported below, being both comprised within the scope of the invention:

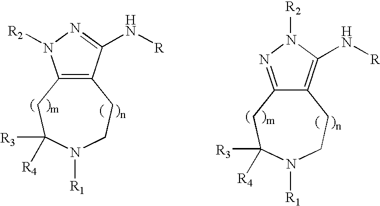

In addition, it also clear that depending upon the nature of the R₃ and R₄ groups and of the meanings of m and n, the following compounds of the invention are thus identified:

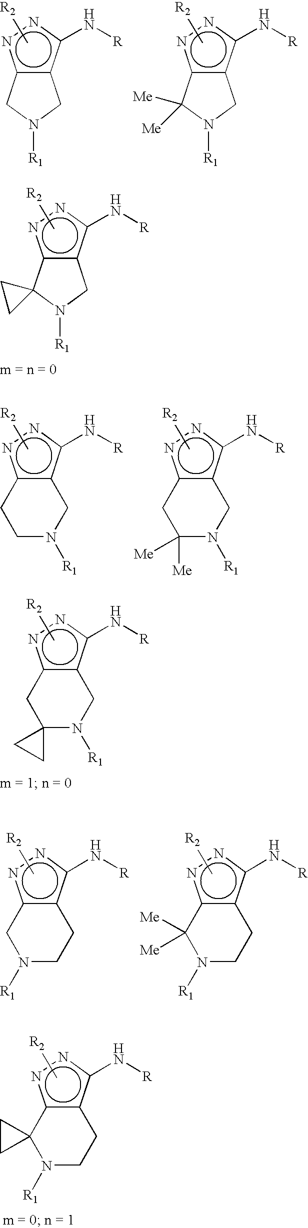

A first class of preferred compounds of the invention is represented by the derivatives of formula (I) wherein R₂ is hydrogen or a group —COOR′, wherein R′ is a straight or branched C₁-C₆ alkyl group.

Even more preferred, within this class, are the compounds of formula (I) wherein & is hydrogen or a group —COOR′ wherein R′ is ethyl.

Another class of preferred compounds of the invention is represented by the derivatives of formula (I) wherein R₁ is an optionally substituted heterocycle selected from pyrimidine, thiazole or benzothiazole.

Even more preferred, within this class, are the compounds of formula (I) wherein R₁ is a pyrimidine ring optionally substituted by one or more groups selected from halogen, heterocycles, alkylheterocycles, hydroxyalkylheterocycles, alkoxy, heterocyclyloxy, alkylheterocyclyloxy, alkylthio, alkylsulfonyl, alkylamino, cycloalkylamino, arylamino and arylalkylamino, wherein heterocyclyl, alkyl, cycloalkyl and aryl are as above defined.

Still more preferred, in any one of the above classes, are the derivatives of formula (I) wherein m is 0 or 1 and n is 0.

Specific examples of compounds of formula (I), optionally in the form of pharmaceutically acceptable salts, are reported in the experimental section.

As set forth above, it is a further object of the present invention a process for preparing the pyrazole compounds of formula (I).

Therefore, the compounds of formula (I) and the pharmaceutically acceptable salts thereof may be prepared by a process comprising:
a) reacting under acidic or basic conditions a compound of formula (II) 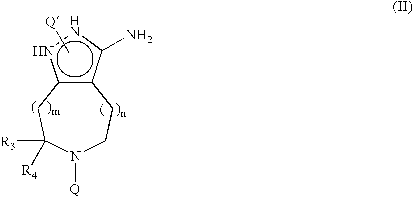
wherein R₃, R₄, m and n are as defined in formula (I), Q represents a suitable nitrogen protecting group and Q′ represents R₂ or a suitable nitrogen protecting group; so as to obtain a compound of formula (III) 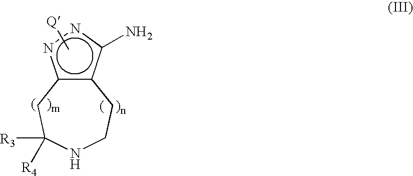
b) reacting the compound of formula (III) with a derivative of formula (IV)
X—R₁ (IV)
wherein R₁ is as defined in formula (I) and X represents a halogen atom or a suitable leaving group, so as to obtain a compound of formula (V) 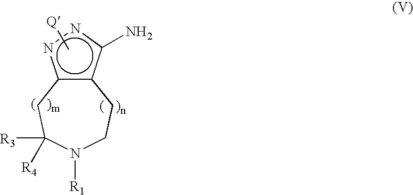
c) reacting the compound of formula (V) with a suitable derivative of formula (VI), (VII), (VIII), (IX), (X) or (XI)
R′COX (VI);
R′OCOX (VII);
R′NCO (VIII);
H₂N—C(═NH)NH₂ (IX);
XSO₂R′ (X);
XSO₂NR′R″ (XI)
wherein R′ and R″ are as defined in formula (I) and X is a halogen atom or a suitable leaving group, so as to obtain the corresponding compound of formula (I) below 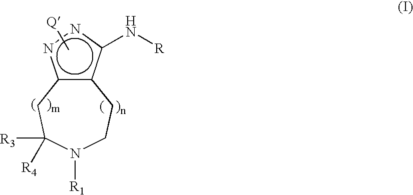
and, optionally,
d) converting it into another compound of formula (I) or into a pharmaceutically acceptable salt thereof.

According to step (a) of the process, the compound of formula (II) is treated under acidic or basic conditions, in the presence of a suitable solvent, for instance dichloromethane or 1,4-dioxane, at room temperature, so as to get the compound of formula (III).

The choice of using basic or acidic conditions is driven by the nature of the Q and Q′ groups, as this reaction enables selective deprotection of the Q group at the non-pyrazole nitrogen atom without affecting the Q′ group at the pyrazole nitrogen atom.

Preferably, within the compound of formula (II), Q represents the group tert-butoxycarbonyl (boc) whereas Q′ is a hydrogen atom or a group R₂ of formula —COOR′ wherein R′ is lower alkyl for instance ethyl.

In this case, the compound of formula (II) may be treated under acidic conditions, for instance in the presence of trifluoroacetic or hydrochloric acid, so as get cleavage of the Q group to the corresponding compound of formula (III).

Clearly, by working under acidic conditions, the compound of formula (III) will be in the form of an addition salt, e.g. as a trifluoroacetate or chloridrate salt.

According to step (b) of the process, the compound of formula (III) is then reacted with a suitable derivative of formula (IV) so as to get the compound of formula (V). The reaction is carried out under basic conditions, for instance in the presence of a suitable alkali carbonate, e.g. potassium carbonate, or of a tertiary base, e.g. diisopropylethylamine.

The reaction is carried out in a suitable solvent such as dimethylsulfoxide (DMSO), dimethylformamide, acetonitrile, isopropanol or n-butanol, at a temperature ranging from room temperature to refluxing temperature. Preferably, within the compound of formula (IV), X represents a chlorine atom or a suitable leaving group such as an alkylsulfonyl or arylsulfonyl group.

According to step (c) of the process, the compound of formula (V) is then reacted with any one of the alternative compounds of formula from (VI) to (XI), so as to get the corresponding compound of formula (I) being properly functionalized at the amino position.

From the above, it is clear to the skilled person that by reacting the compound of formula (V): with a derivative of formula (VI), compounds having R as a —COR′ group may be obtained; with a derivative of formula (VII), compounds having R as a —COOR′ group may be obtained; with a derivative of formula (VIII), compounds having R as a —CONHR′ group may be obtained; with a derivative of formula (IX), compounds having R as a

—C(═NH)NH₂ group may be obtained; with a derivative of formula (X), compounds having R as a —SO₂R′ group may be obtained and; finally, with a derivative of formula (XI), compounds having R as a —SO₂NR′R″ group may be obtained.

Any of the above reactions and operative conditions thereof are widely known in the art as they allow to obtain a variety of carboxamido, carbamato, ureido, amidino, sulfonamido or sulfonylureido derivatives.

The compounds of formula (I) thus obtained may be then converted, according to step (d) of the process, in a variety of other compounds of formula (I) of the invention, and/or into pharmaceutically acceptable salts thereof, by working according to conventional methods.

As an example, the compounds of formula (I) wherein Q′ stands for a nitrogen protecting group may be easily deprotected according to known methods, for instance under acidic or basic conditions as the case may be, so as to obtain the corresponding compounds wherein R₂ stands for hydrogen; their subsequent functionalization at the pyrazole nitrogen atom may then allow to insert any desired R₂ group.

Likewise, as an additional example of conversion according to step (d), the amidino compounds of formula (I) wherein R stands for —C(═NH)NH₂ may be easily converted into a variety of derivatives wherein R is a —C(═NH)NHR′ group, by means of known methods, for instance in the presence of a suitable amino derivative R′NH₂, wherein R′ is as above defined.

From all of the above, it is also clear that any optional substituent that is part of R₁, R′ or R″ and that is further susceptible of being converted into another group may also lead to a variety of derivatives.

As a non limiting example, carboxy groups may be converted into a variety of derivatives including esters and amides; carboxamides may undergo reduction to amino derivatives; alkylthio groups may be oxidized to alkylsulfinyl or alkylsulfonyl groups or even replaced by amino or alkoxy groups and derivatives thereof; chlorine atoms may be replaced by amino or alkoxy groups and derivatives thereof; nitro groups can be reduced to amines; amines may be further acylated to carboxamides, and the like.

For a general reference to any one of the above reactions, herewith conveniently grouped into step (d) of the process, and to the operative conditions thereof (for instance including the use of microwave and of given apparatus, according to methods known in the art), see the experimental section.

When preparing the compounds of formula (I) according to any variant of the process, which are all to be intended as within the scope of the present invention, optional functional groups within both the starting materials, the reagents or the intermediates thereof and which could give rise to unwanted side reactions, need to be properly protected according to conventional techniques.

Likewise, the conversion of these latter into the free deprotected compounds may be carried out according to known procedures.

By analogy, pharmaceutically acceptable salts of the compounds of formula (I) or, alternatively, their free compounds from the salts thereof, my be all obtained according to conventional methods.

From all of the above, it is also clear to the person skilled in the art that if a compound of formula (I), prepared according to the above process, is obtained as an admixture of isomers, their separation into the single isomers of formula (I), carried out according to conventional techniques, is still within the scope of the present invention.

The compounds of formula (I) may be also prepared according to an alternative synthetic approach represented below, still to be intended as comprised within the scope of the invention:

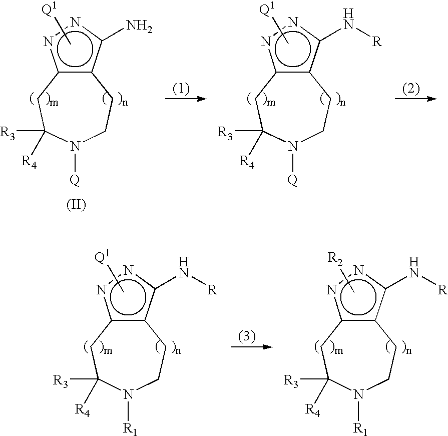

From the above, it is clear to the skilled person that the operative conditions being employed in this latter process are substantially analogous to those of the previous one, with the exception that some steps are carried out according to a different order.

In step (1), in fact the compound of formula (II) is first reacted so as to get the desired amino derivative (—NHR) as per previous step (c); in step (2), the intermediate thus obtained is deprotected at the non-pyrazole nitrogen atom and subsequently reacted with a compound of formula (IV), as per previous step (b); finally, in step (3), the obtained compound is further converted into the derivative of formula (I) as per previous step (d). For a general reference to the above process and operative conditions thereof, see the experimental section.

Very advantageously, the compounds of formula (I) of the invention may be also prepared under solid-phase-synthesis (SPS) conditions, which are typically adopted when preparing libraries of compounds according to combinatorial chemistry techniques.

Therefore, it is a further object of the invention, a process for preparing the compounds of formula (I) of the invention which comprises:
e) hydrolyzing under acidic or basic conditions the compound of formula (I) being obtained in previous step (c) wherein R, R₁, R₃, R₄, m and n have the above reported meanings and Q′ is a suitable pyrazole nitrogen protecting group 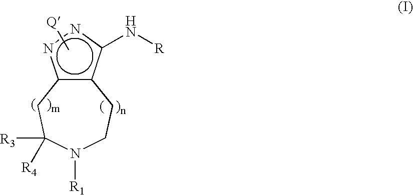
and reacting the thus obtained compound bearing a hydrogen atom in place of Q′ in the presence of a suitable polymeric resin (P), so as to obtain the resin supported compound of formula (XII) 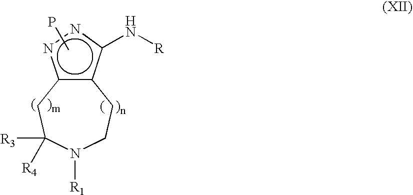
f) optionally converting the compound of formula (XII) into another compound of formula (XII), and
g) cleaving the polymeric resin so as to obtain the desired compound of formula (I) and, whenever desired, converting it into a pharmaceutically acceptable salt thereof.

According to step (e) of the process, the compound of formula (I) being obtained in step (c) is first deprotected at the pyrazole nitrogen atoms according to conventional means, for instance under acidic or basic hydrolysis conditions.

The obtained derivative is then conveniently anchored to an inert polymeric support (P) such as, for instance, 2-chloro-trityl chloride resin, trityl chloride resin, p-nitrophenyl carbonate Wang resin, or an isocyanate polystyrenic resin, which are all conventionally known in this field.

Typically, for instance when Q′ is a carboxyester group —COOR′, the cleavage of the Q′ group may occur under basic conditions and in the presence of the polymeric resin, so as to allow loading of the pyrazole intermediate onto the solid phase.

The reaction may be carried out in the presence of a slight excess of a suitable base, for instance an amine such as diisopropylethylamine (DIPEA), triethylamine (TEA), 1,8-diazabiciclo[5.4.0]undec-7-ene (DBU) or 2-tert-butylimino-2-diethylamino-1,3-dimethylperhydro-1,3,2-diaza-phosphorine, in a suitable solvent such as, for instance, dichloromethane, chloroform, methanol, tetrahydrofuran, dimethylformamide, dimethylacetamide, and the like.

The reaction may be performed by adding to a suspension of the resin, the base and the compound of formula (I) to be supported, under stirring and at a temperature ranging from room temperature to about 55° C. for a suitable time, for instance up to 96 hours.

The polymer supported compound of formula (XII) thus obtained may be reacted, in step (f), so as to give rise to a variety of derivatives, substantially as set forth above in step (d) of the process.

Finally, in step (g), the compound of formula (XII) is cleaved from the resin to which it is supported; resin cleavage may be carried out for instance, in the presence of trifluoroacetic acid so as to yield the desired compound of formula (I). The compound of formula (3) is thus suspended in a solution of 5-95% of trifluoroacetic acid in dichloromethane, and the mixture is stirred at room temperature for a suitable time, for instance from a few minutes to about 3 hours.

Alternatively, resin cleavage may be also carried out under basic conditions, for instance in the presence of aqueous potassium or sodium hydroxide and in the presence of a suitable co-solvent such as methanol, ethanol, dimethylformamide, 1,4-dioxane or acetonitrile, so as to yield the desired compound of formula (I). The compound of formula (XII) is thus suspended in a solution of 35% of sodium or potassium hydroxide, for instance in methanol, by working under mild operative conditions at temperatures ranging from about 5° C. to about 60° C. and for a time varying from about 2 hours to about 7 days.

The compound of formula (II), as a starting material of each one of the above processes, comprehensive of any variant thereof, is known or can be easily obtained according to known methods.

In particular, the compound of formula (II) wherein Q is tert-butoxycarbonyl (boc) and Q′ is ethoxycarbonyl (—COOEt), as per the formula below 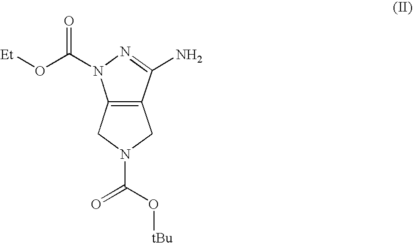
is known and may be prepared as described in the aforementioned international patent application WO 02/12242. Likewise, any other derivative of formula (I) may be also prepared, by analogy, as described in WO 02/12242.

In addition, the compounds of formula (IV) and of formula from (VI) to (XI), the polymeric resin as well as any other reactant of the processes of the invention, is known or can be prepared according to known methods.

As formerly indicated, the compounds of formula (I) may be conveniently prepared according to parallel synthesis or combinatorial chemistry techniques widely known in the art, by accomplishing the aforementioned reactions between the several intermediates in a serial manner and by working under SPS (Solid Phase Synthesis) or Solution Phase Synthesis conditions.

Accordingly, it is a further object of the present invention a library of two or more pyrazole derivatives represented by formula (I) 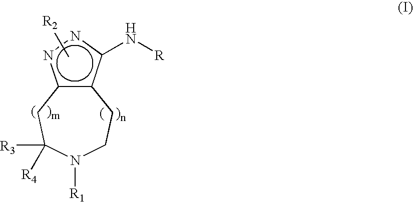
wherein
R is a hydrogen atom or a group selected from —COR′, —COOR′, —CONHR′, —C(═NH)NHR′, —SO₂R′ or —SO₂NR′R″;
R₁ is an optionally substituted 5 or 6 membered heterocyclic group with from 1 to 3 heteroatoms or heteroatomic groups selected from N, NR′, O or S, optionally benzocondensed;
R₂ is hydrogen or it is selected from the group consisting of R′, —COR′, —COOR′, —CONR′R″ or —S(O)_{q}R′;
R₃ and R₄ are both hydrogen atoms or methyl groups or, together with the carbon atom to which they are attached, form a cyclopropyl group;
R′ and R″ are, the same or different and independently in each of the above occasions, a hydrogen atom or an optionally substituted group selected from straight or branched C₁-C₆ alkyl, C₃-C₆ cycloalkyl, aryl, aryl C₁-C₆ alkyl heterocyclyl or heterocyclyl C₁-C₆ alkyl;
m and n are 0 or 1, provided that they are not both 1;
q is 0 or an integer from 1 to 2;
and the pharmaceutically acceptable salts thereof.

From all of the above, it is clear to the skilled person that once a library of pyrazole derivatives is thus prepared, for instance consisting of several hundreds of compounds of formula (I), the said library can be very advantageously used for screening towards given kinases, as formerly reported.

See, for a general reference to libraries of compounds and uses thereof as tools for screening biological activities, J. Med. Chem. 1999, 42, 2373-2382; and Bioorg. Med. Chem. Lett. 10 (2000), 223-226.

Pharmacology

The compounds of formula (I) are active as protein kinase inhibitors and are therefore useful, for instance, to restrict the unregulated proliferation of tumor cells.

In therapy, they may be used in the treatment of various tumors, such as those formerly reported, as well as in the treatment of other cell proliferative disorders such as psoriasis, vascular smooth cell proliferation associated with atherosclerosis and post-surgical stenosis and restenosis and in the treatment of Alzheimer's disease.

The inhibiting activity of putative Cdk/Cyclin inhibitors and the potency of selected compounds is determined through a method of assay based on the use of the SPA technology (Amersham Pharmacia Biotech).

The assay consists of the transfer of radioactivity labelled phosphate moiety by the kinase to a biotinylated substrate. The resulting 33P-labelled biotinylated product is allowed to bind to streptavidin-coated SPA beads (biotin capacity 130 pmol/mg), and light emitted is measured in a scintillation counter.

Inhibition Assay of Cdk2/Cyclin A Activity

Kinase reaction: 4 μM in house biotinylated histone H1 (Sigma # H-5505) substrate, 10 μM ATP (0.1 microCi P³³γ-ATP), 4.2 ng Cdk2/Cyclin A complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 30 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate. After 20 min. incubation for substrate capture, 100 μl 5M CsCl were added to allow statification of beads to the top of the plate and let stand 4 hours before radioactivity counting in the Top-Count instrument

IC50 determination: inhibitors were tested at different concentrations ranging from 0.0015 to 10 μM. Experimental data were analyzed by the computer program GraphPad Prizm using the four parameter logistic equation:
y=bottom+(top−bottom)/(1+10ˆ((log IC50−x)*slope))
where x is the logarithm of the inhibitor concentration, y is the response; y starts at bottom and goes to top with a sigmoid shape.
Ki Calculation:
Experimental method: Reaction was carried out in buffer (10 mM Tris, pH 7.5, 10 mM MgCl₂, 0.2 mg/ml BSA, 7.5 mM DTI) containing 3.7 nM enzyme, histone and ATP (constant ratio of cold/labeled ATP 1/3000). Reaction was stopped with EDTA and the substrate captured on phosphomembrane (Multiscreen 96 well plates from Millipore). After extensive washing, the multiscreen plates are read on a top counter. Control (time zero) for each ATP and histone concentrations was measured.
Experimental design: Reaction velocities are measured at different four ATP, substrate (histone) and inhibitor concentrations. An 80-point concentration matrix was designed around the respective ATP and substrate Km values, and the inhibitor IC50 values (0.3, 1, 3, 9 fold the Km or IC50 values). A preliminary time course experiment in the absence of inhibitor and at the different ATP and substrate concentrations allow the selection of a single endpoint time (10 min) in the linear range of the reaction for the Ki determination experiment.
Kinetic parameter estimates: Kinetic parameters were estimated by simultaneous nonlinear least-square regression using [Eq.1] (competitive inhibitor respect to ATP, random mechanism) using the complete data set (80 points):

$$v = \frac{Vm \cdot A \cdot B}{α \cdot Ka \cdot Kb + α \cdot Ka \cdot B + a \cdot Kb \cdot A + A \cdot B + α \cdot \frac{Ka}{Ki} \cdot I \cdot ( Kb + \frac{B}{β} )} \qquad [ Eq . 1 ]$$

where A=[ATP], B=[Substrate], I=[inhibitor], Vm=maximum velocity, Ka, Kb, Ki the dissociation constants of ATP, substrate and inhibitor respectively. α and β the cooperativity factor between substrate and ATP binding and substrate and inhibitor binding respectively.

In addition the selected compounds are characterized on a panel of ser/threo kinases strictly related to cell cycle (Cdk2/Cyclin E, Cdk1/cyclin B1, Cdk5/p25, Cdk4/Cyclin D1), and also for specificity on MAPK, PKA, EGFR, IGF1-R, Aurora-2 and Akt.

Inhibition Assay of Cdk2/Cyclin E Activity

Kinase reaction: 10 μM in house biotinylated histone H1 (Sigma # H5505) substrate, 30 μM ATP (0.3 microCi P³³γ-ATP), 4 ng GST-Cdk2/Cyclin B complex, inhibitor in a final volume of 30 μl buffer (TRIS HCl 10 nM pH 7.5, MgCl₂ 10 mM, DTT 7.5 mM+0.2 mg/ml BSA) were added to each well of a 96 U bottom. After 60 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the plate and let stand 4 hours before radioactivity counting in the Top-Count instrument

IC50 determination: see above

Inhibition Assay of Cdk1/Cyclin B1 Activity

Kinase reaction: 4 μM in house biotinylated histone H1 (Sigma # H-5505) substrate, 20 μM AT P (0.2 microCi P³³γ-ATP), 3 ng Cdk1/Cyclin B complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 20 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of Cdk5/p25 Activity

The inhibition assay of Cdk5/p25 activity was performed according to the following protocol.

Kinase reaction: 10 μM biotinylated histone H1 (Sigma # H-5505) substrate, 30 μM ATP (0.3 microCi P³³γ-ATP), 15 ng CDK5/p25 complex, inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 30 min at r.t incubation, reaction was stopped by 100 μl PBS+32 mM EDTA+0.1% Triton X-100+500 μM ATP, containing 1 mg SPA beads. Then a volume of 110 μl is transferred to Optiplate.

After 20 mm incubation for substrate capture, 100 μl 5M CsCl were added to allow statification of beads to the top of the plate and let stand 4 hours before radioactivity counting in the Top-Count instrument.

IC50 determination: see above

Inhibition Assay of Cdk4/Cyclin D1 Activity

Kinase reaction: 0.4 μM mouse GST-Rb (769-921) (# sc 4112 from Santa Cruz) substrate, 10 μM ATP (0.5 μCi P³³γ-ATP), 100 ng of baculovirus expressed GST-Cdk4/Cyclin D1, suitable concentrations of inhibitor in a final volume of 50 μl buffer (TRIS HCl 10 mM pH 7.5, MgCl₂ 10 mM 7.5 mM DTT+0.2 mg/ml BSA) were added to each well of a 96 U bottom well plate. After 40 min at 37° C. incubation, reaction was stopped by 20 μl EDTA 120 mM.

Capture: 60 μl were transferred from each well to MultiScreen plate, to allow substrate binding to phosphocellulose filter. Plates were then washed 3 times with 150 μl/well PBS Ca⁺⁺/Mg⁺⁺ free and filtered by MultiScreen filtration system.

Detection: filters were allowed to dry at 37° C., then 100 μl/well scintillant were added and ³³P labeled Rb fragment was detected by radioactivity counting in the Top-Count instrument.

IC50 determination: see above

Inhibition Assay of MAPK Activity

Kinase reaction: 10 μM in house biotinylated MBP (Sigma # M-1891) substrate, 15 μM ATP (0.15 microCi P³³γ-ATP), 30 ng GST-MAPK (Upstate Biotechnology # 14-173), inhibitor in a final volume of 30 μl buffer (TRIS HCl 10 mM pH 7.5, MgCl₂ 10 mM, DTT 7.5 mM+0.2 mg/ml BSA) were added to each well of a 96 U bottom. After 30 min at r.t incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of PKA Activity

Kinase reaction: 10 μM in house biotinylated histone H1 (Sigma # H-5505) substrate, 10 μM ATP (0.2 microM P³³γ-ATP), 0.45 U PKA (Sigma # 2645), inhibitor in a final volume of 30 μl buffer (TRIS HCl 10 mM pH 75, MgCl₂ 10 mM, DTT 7.5 mM+0.2 mg/ml BSA) were added to each well of a 96 U bottom. After 90 min at r.t incubation, reaction was stopped by 100 μl PBS+32 mM EDTA+0.1% Triton X-100+500 μM ATP, containing 1 mg SPA beads. Then a volume of 110 μl is transferred to Optiplate. After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of EGFR Activity

Kinase reaction: 10 μM in house biotinylated MBP (Sigma # M-1891) substrate, 2 μM ATP (0.04 microCi P³³γ-ATP), 36 ng insect cell expressed GST-EGFR, inhibitor in a final volume of 30 μl buffer (Hepes 50 mM pH 7.5, MgCl₂ 3 mM MnCl₂ 3 mM, DTT 1 mM NaVO₃ 3 μM+0.2 mg/ml BSA) were added to each well of a 96 U bottom. After 20 min at r.t. incubation, reaction was stopped by 100 μl PBS+32 mM EDTA+0.1% Triton X-100+500 μM ATP, containing 1 mg SPA beads. Then a volume of 110 μl is transferred to Optiplate.

After 20 min. incubation for substrate capture, 100 μl 5M CsCl were added to allow statification of beads to the top of the Optiplate and let stand 4 hours before radioactivity counting in the Top-Count instrument.

IC50 determination: see above

Inhibition Assay of IGF1-R Activity

The inhibition assay of IGF1-R activity was performed according to the following protocol.

Kinase reaction: 10 μM biotinylated MBP (Sigma cat # M-1891) substrate, 0-20 μM inhibitor, 6 μM ATP, 1 microCi ³³P-ATP, and 22.5 ng GST-IGF1-R (pre-incubated for 30 min at room temperature with cold 60 μM cold ATP) in a final volume of 30 μl buffer (50 mM HEPES pH 7.9, 3 mM MnCl₂, 1 mM DTT, 3 μM NaVO₃) were added to each well of a 96 U bottom well plate. After incubation for 35 min at room temperature, the reaction was stopped by addition of 100 μl PBS buffer containing 32 mM EDTA, 500 μM cold ATP, 0.1% Triton X100 and 10 mg/ml streptavidin coated SPA beads. After 20 min incubation, 110 μl of suspension were withdrawn and transferred into 96-well OPTIPLATEs containing 100 μl of 5M CsCl. After 4 hours, the plates were read for 2 min in a Packard TOP-Count radioactivity reader.

Inhibition Assay of Aurora-2 Activity

Kinase reaction: 8 μM biotinylated peptide (4 repeats of LRRWSLG), 10 μM ATP (0.5 uCi P³³γ-ATP), 15 ng Aurora2, inhibitor in a final volume of 30 μl buffer (HEPES 50 mM pH 7.0, MgCl₂ 10 mM, 1 mM DTT, 0.2 mg/ml BSA, 3 μM orthovanadate) were added to each well of a 96 U bottom well plate. After 30 minutes at room temperature incubation, reaction was stopped and biotinylated peptide captured by adding 100 μl of bead suspension.

Stratification: 100 μl of CsCl2 5 M were added to each well and let stand 4 hour before radioactivity was counted in the Top-Count instrument.

IC50 determination: see above

Inhibition Assay of Cdc7/dbf4 Activity

The inhibition assay of Cdc7/dbf4 activity was performed according to the following protocol.

The Biotin-MCM2 substrate is trans-phosphorylated by the Cdc7/Dbf4 complex in the presence of ATP traced with γ³-ATP. The phosphorylated Biotin-MCM2 substrate is then captured by Streptavidin-coated SPA beads and the extent of phosphorylation evaluated by β counting.

The inhibition assay of Cdc7/dbf4 activity was performed in 96 wells plate according to the following protocol.

To each well of the plate were added:
10 μl substrate (biotinylated MCM2, 6 μM final concentration)
10 μl enzyme (Cdc7/Dbf4, 12.5 nM final concentration)
10 μl test compound (12 increasing concentrations in the nM to μM range to generate a dose-response curve)
10 μl of a mixture of cold ATP (10 μM final concentration) and radioactive ATP (1/2500 molar ratio with cold ATP) was then used to start the reaction which was allowed to take place at 37° C.

Substrate, enzyme and ATP were diluted in 50 mM HEPES pH 7.9 containing 15 mM MgCl₂, 2 mM DTT, 3 μM NaVO₃, 2 mM glycerophosphate and 0.2 mg/ml BSA. The solvent for test compounds also contained 10% DMSO.

After incubation for 20 minutes, the reaction was stopped by adding to each wen 100 μl of PBS pH 7.4 containing 50 mM EDTA, 1 mM cold ATP, 0.1% Triton X100 and 10 mg/ml streptavidin coated SPA beads.

After 15 minutes of incubation at room temperature to allow the biotinylated MCM2-streptavidin SPA beads interaction to occur, beads were trapped in a 96 wells filter plate (Unifilter® GF/B™) using a Packard Cell Harvester (Filtermate), washed with distilled water and then counted using a Top Count (Packard).

Counts were blank-subtracted and then the experimental data (each point in triplicate) were analyzed for IC50 determination using a non-linear regression analysis (Sigma Plot).

Given the above inhibition assays, the compounds of formula (I) of the invention resulted to possess a remarkable kinase inhibitory activity.

See, as an example, the following experimental data (IC₅₀) of two representative compounds of the invention of formula (I) being tested against Cdk2/Cyclin A:
3-(4-tert-butyl-benzamido)-5-[(2-propylamino)pyrimidin-4-yl]-4,6-dihydropyrrolo[3,4-c]pyrazole (IC₅₀ 0.22 μM); and
3-(4-tert-butyl-benzamido)-5-[2-(N-morpholino)pyrimidin-4-yl]-4,6-dihydropyrrolo[3,4-c]pyrazole (IC₅₀ 0.34 μM).

Surprisingly, their inhibitory activity resulted to be markedly superior than that of the prior art compounds of WO 02/12242 (reference compounds), being used for comparative purpose and tested against Cdk2/Cyclin A as above reported.

Reference Compound:

N-{5-(pyridine-4-carbonyl)-4,6-dihydropyrrolo[3,4-c]pyrazol-3-yl}-4-tertbutylbenzamide (IC₅₀>10 μM), [see WO 02/12242 on page 143, lines 29-32]; and
N-{5-(pyrazine-2-carbonyl)-4,6-dihydropyrrolo[3,4-c]pyrazol-3-yl}-4-tertbutylbenzamide (IC₅₀ 2.5 μM), [see WO 02/12242 on page 144, lines 5-8].

So far, the novel compounds of the invention are unexpectedly endowed with a cdk inhibitory activity significantly higher than that of the structurally closest prior art compounds of WO 02/12242 and are thus particularly advantageous, in therapy, against proliferative disorders associated with an altered cell cycle dependent kinase activity.

The compounds of formula (I) of the present invention, suitable for administration to a mammal, e.g. to humans, can be administered by the usual routes and the dosage level depends upon the age, weight, conditions of the patient and the administration route.

For example, a suitable dosage adopted for oral administration of a compound of formula (I) may range from about 10 to about 500 mg pro dose, from 1 to 5 times daily. The compounds of the invention can be administered in a variety of dosage forms, e.g. orally, in the form of tablets, capsules, sugar or film coated tablets, liquid solutions or suspensions; rectally in the form of suppositories; parenterally, e.g. intramuscularly, or by intravenous and/or intrathecal and/or intraspinal injection or infusion.

In addition, the compounds of the invention can be administered either as single agents or, alternatively, in combination with known anticancer treatments such as radiation therapy or chemotherapy regimen in combination with cytostatic or cytotoxic agents, antibiotic-type agents, alkylating agents, antimetabolite agents, hormonal agents, immunological agents, interferon-type agents, cyclooxygenase inhibitors (e.g. COX-2 inhibitors), metallomatrixprotease inhibitors, telomerase inhibitors, tyrosine kinase inhibitors, anti-growth factor receptor agents, anti-HER agents, anti-EGFR agents, anti-angiogenesis agents, farnesyl transferase inhibitors, ras-raf signal transduction pathway inhibitors, cell cycle inhibitors, other cdks inhibitors, tubulin binding agents, topoisomerase I inhibitors, topoisomerase II inhibitors and the like, optionally within liposomal formulations thereof.

If formulated as a fixed dose, such combination products employ the compounds of this invention within the dosage range described above and the other pharmaceutically active agent within the approved dosage range.

Compounds of formula (I) may be used sequentially with known anticancer agents when a combination formulation is inappropriate.

The present invention also includes pharmaceutical compositions comprising a compound of formula (I) or a pharmaceutically acceptable salt thereof in association with a pharmaceutically acceptable excipient (which can be a carrier or a diluent).

The pharmaceutical compositions containing the compounds of the invention are usually prepared following conventional methods and are administered in a pharmaceutically suitable form.

For example, the solid oral forms may contain, together with the active compound, diluents, e.g. lactose, dextrose, saccharose, sucrose, cellulose, corn starch or potato starch; lubricants, e.g. silica, talc, stearic, magnesium or calcium stearate, and/or polyethylene glycols; binding agents, e.g. starches, arabic gum, gelatin, methylcellulose, carboxymethylcellulose or polyvinyl pyrrolidone; disaggregating agents, e.g. a starch, alginic, alginates or sodium starch glycolate; effervescing mixtures; dyestuffs; sweeteners; wetting agents such as lecithin, polysorbates, laurylsulfates; and, in general, non-toxic and pharmacologically inactive substances used in pharmaceutical formulations. Said pharmaceutical preparations may be manufactured according to known techniques, for example by means of mixing, granulating, tabletting, sugar-coating, or film-coating processes.

The liquid dispersions for oral administration may be, e.g., syrups, emulsions and suspensions.

The syrups may contain as carrier, for example, saccharose or saccharose with glycerin and/or mannitol and/or sorbitol.

The suspensions and the emulsions may contain as carrier, for example, a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and, if desired, a suitable amount of lidocaine hydrochloride. The solutions for intravenous injections or infusions may contain as a carrier, for example, sterile water or preferably they may be in the form of sterile, aqueous, isotonic saline solutions or they may contain as a carrier propylene glycol.

The suppositories may contain together with the active compound a pharmaceutically acceptable carrier, e.g. cocoa butter, polyethylene glycol, a polyoxyethylene sorbitan fatty ester surfactant or lecithin.

The following examples are herewith intended to better illustrate the present invention without posing any limitation to it.

### General Methods

HPLC Conditions

Instrumentation: Waters 2795 HPLC system equipped with a 996 Waters PDA detector and Micromass mod. ZQ single quadrupole mass spectrometer, equipped with an electrospray (ESI) ion source.

Chromatographic condition: RP18 Waters X Terra (4.6×50 mm, 3.5 μm) column; Mobile phase A was ammonium acetate 5 mM buffer (pH 5.5 with acetic acid/acetonitrile 95:5), and Mobile phase B was H₂O/acetonitrile (5:95). Gradient from 10 to 90% B in 8 minutes, hold 90% B 2 minutes. UV detection at 220 nm and 254 nm. Flow rate 1 ml/min. Injection volume 10 μl. Full scan, mass range from 100 to 800 amu. Capillary voltage was 2.5 KV; source temperature was 120° C.; cone was 10 V. Retention times (HPLC r.t.) are given in minutes at 220 nm or at 254 nm. Mass are given as m/z ratio.

As formerly indicated, several compounds of formula (I) of the invention have been synthesized in parallel, according to combinatorial or parallel chemistry techniques and purified, when necessary, using parallel purification techniques.

In this respect, some compounds thus prepared have been conveniently and unambiguously identified with HPLC retention time and mass.

### EXAMPLE 1

### Preparation of 3-amino-5-tert-butyloxycarbonyl-1-ethoxycarbonyl-4,6-dihydropyrrolo[3,4-c]pyrazole

A solution of ethyl chlorocarbonate (8.9 ml, 93 mmol) in THF (250 ml) was added slowly to a mixture of 3-amino-4,6-dihydro-1H-pyrrolo[3,4-c]pyrazole-5-carboxylic acid tert-butyl ester (20 g, 89 mmol) and diisopropylethylamine (DIEA, 92 ml, 528 mmol) in THF (500 ml) at 05° C. The reaction was kept at the same temperature for two hours then allowed to reach room temperature and stirred overnight. The obtained mixture was evaporated to dryness under vacuum. The resulting residue was extracted with ethyl acetate and water. The organic phase was separated, dried over sodium sulfate and evaporated to dryness. The mixture was purified by flash-chromatography (eluent:ethyl acetate/cyclohexane 4/6 to 7/3) to give 19 g (72% yield) of the title compound as a white sold.

1H-NMR (400 MHz, DMSO-δ6) ppm: 10.06 (s, broad signal, 2H), 4.4-4.05 (m, 6H), 1.27 (t, 3H) 1 (2, 9H).

### EXAMPLE 2

### Preparation of 3-amino-1-ethoxycarbonyl-4,6-dihydropyrrolo[3,4-c]pyrazole

3-Amino-5-tert-butyloxycarbonyl-1-ethoxycarbonyl-4,6-dihydropyrrolo[3,4-c]pyrazole (1 g, 3.37 mmol) was treated with 4N H in 1,4-dioxane (20 eq.) and dichloromethane (50 ml) at mom temperature for about 2 hours. After evaporation of the solvents, the crude material was triturated with diethyl ether, filtered and dried under vacuum to yield the title compound as the hydrochloric acid salt which was used without any further purification (colourless solid, quantitative yield).

1H-NMR (400 MHz, DMSO-δ6) ppm: 10.06 (s, broad signal, 2M), 4.38-4.08 (m, 6H), 1.27 (t, 31).

By operating in an analogous way the following compounds were also obtained:
3-[4-(morpholin-1-yl)-benzamido]-1-ethoxycarbonyl-4,6-dihydropyrrolo[3,4-c]pyrazole

1H-NMR (400 MT, DMSO-δ6) ppm: 11.33 (s, broad signal, 2H), 10.10 (m, broad signal, 1H), 7.97 (m, 2M), 7.02 (m, 2H), 4.49-4.42 (m, 6H), 3.75-3.29 (m, 8M), 1.36 (t, 3H);
3-[4-(N-methyl-piperazin-1-yl)-benzamido]-1-ethoxycarbonyl-4,6-dihydropyrrolo[3,4-c]pyrazole

1H-NMR (400 MHz, DMSO-δ6) ppm: 11.38 (s, broad signal, 2H), 10.26 (m, broad signal, 1H), 8.00 (m, 2H), 7.10 (m, 2H), 4.56-4.42 (m, H), 4.06-3.22(m, 8H), 2.83 (m, 3H), 1.37 (t, 3H).

### EXAMPLE 3

### Preparation of 3-amino-1-ethoxycarbonyl-5-[(2-methylthio)pyrimidin-4-yl]-4,6-dihydropyrrolo[3,4-c]pyrazole

2-Methylthio-4-chloropyrimidine (0.019 ml, 0.16 mmol) and K₂CO₃ (33 mg, 0.24 mmol) were added to a solution of 3-amino-1-ethoxycarbonyl-4,6-dihydropyrrolo[3,4-c]pyrazole (50 mg, 0.16 mmol) in DMSO (1 ml), being prepared according to the example 2.

The mixture was heated at 90° C. for 4 hours. Water (30 ml) was added and the mixture was extracted with ethyl acetate (2×20 ml). The organic layers were combined, washed with brine, dried over sodium sulfate, filtered and dried under vacuum. The crude material was purified by flash chromatography on silica gel, using cyclohexane:ethyl acetate as eluent, to yield the title compound as a white solid (25 mg, yield 80%).

1H-NMR (400 MHz, DMSO-δ6) ppm: 8.09-8.02 (m, 1H), 6.34-6.16 (m, 1H), 4.77-4.21 (m, 4H), 4.33-4.21 (q, 2H, J=7.07 Hz), 2.44 (s, 3H), 1.33-1.23 (t, 3H, J=7.07 Hz).

### EXAMPLE 4

### Preparation of 3-amino-1-ethoxycarbonyl-5-[(2-chloro)pyrimidin-4-yl]-4,6-dihydropyrrolo[3,4-c]pyrazole

2,4-Dichloropyrimidine (4.11 g, 18.3 mmol) and diisopropylethyl amine (9.8 ml, 54.9 mmol) were added to a suspension of 3-amino-1-ethoxycarbonyl-4,6-dihydropyrrolo[3,4-c]pyrazole dihydrochloride (4.89 mg, 18.3 mmol) in isopropanol (iPrOH, 300 ml), being prepared according to the example 2.

The mixture was heated at reflux for 7 hours and then it was allowed to cool to room temperature. The title compound was obtained as a beige powder upon filtration of the reaction mixture and it was used without any further purification (5.27 g).

1H-NMR (400 MHz, DMSO-δ6) ppm: 6.6 (d, 1H), 6.45 (d, 1H), 5.77 (s, bs, 2H), 4.65 (m, 2H), 4.27 (m, 4H), 1.25 (t, 3H).

By operating in an analogous way the following compounds were also obtained.
3-[4-morpholin-1-yl)-benzamido]-1-ethoxycarbonyl-5-[(2-chloro)pyrimidin-4-yl]-4,6-dihydropyrrolo[3,4-c]pyrazole

1H-NMR (400 MHz, DMSO-δ6) ppm: 11.22 (s, 1H), 8.13 (d, 1H), 7.98 (d, 2H), 6.96 (d, 2M), 6.62(d, 1H), 4.8-4.6 (m, 4H), 4.4 (q, 2H), 3.72 (m, 4H), 3.25 (m, 4H), 2.35 (t, 3H).
3-[4-(N-methyl-piperazin-1-yl)-benzamido]-1-ethoxycarbonyl-5-[(2-chloro)pyrimidin-4-yl]-4,6-dihydropyrrolo[3,4-c]pyrazole

[M+H]⁺ 511.19; r.t. 4.11 min.

### EXAMPLE 5

### Preparation of 3-(tert-butyl-benzamido)-1-ethoxycarbonyl-5-[(2 methylthio)pyrimidin-4-yl]-4,6-dihydropyrrolo[3,4-c]pyrazole

Tri-(2-aminoethyl)-amine polystirene (6 g, 18.7 mmol) was added to a solution of 3-amino-1-ethoxycarbonyl-5-[(2-methylthio)pyrimidin-4-yl]-4,6-dihydropyrrolo[3,4-c]pyrazole (1.2 g, 3.74 mmol) in dry THF (120 ml). 4-Tert-butyl-benzoyl chloride (6 mmol) was added dropwise to the suspension. The mixture was stared at room temperature for 24 hours. The suspension was filtered, washed with tetrahydrofuran and dichloromethane. After evaporation of the solvents, water was added and the mixture was extracted with ethyl acetate. The organic layers were combined, washed with brine, dried over sodium sulfate, filtered and dried under vacuum. The crude material was purified by flash chromatography on silica gel, using cyclohexane:ethyl acetate as eluent, to yield the title compound (1.07 g, 70%).

1H-NMR (400 MHz, DMSO-δ6) ppm: 11.68-11.19 (m, 1H), 8.12-8.24 (m, 1H), 8.04-7.97 (m, 3H), 7.54-7.49 (m, 2H), 6.41-6.27 (m, 1H), 4.92-4.58 (m, 4H), 4.50-4.32 (q, 2H, 3=7.07 Hz), 2.45 (s, 3M), 1.43-1.26 (m, 12H).

By operating in an analogous way the following compounds were also obtained:
1-ethoxycarbonyl-3-(4-fluoro-benzamido)-5-[(2-methylthio)pyrimidin-4-yl]-4,6-dihydropyrrolo[3,4-c]pyrazole (71% yield)

1H-NMR (400 MHz, DMSO-δ6) ppm: 11.58 (m, 1H), 8.18-8.10 (m, 2H), 8.09-8.03 (m, 1H), 7.37-7.29(m, 211, 6.38-6.27(m, 1H), 4.92-4.58 (m, 4H), 4.46-4.37 (q, 2H, J=7.07 Hz), 2.45 (s, 3H), 1.41-1.31 (t 3H, J=7.07 Hz);
1-ethoxycarbonyl-3-(4-fluoro-benzamido)-5-[(2-methanesulfonyl)pyrimidin-4-yl]-4,6-dihydropyrrolo[3,4-c]pyrazole

1H-NMR (400 MHz, DMSO-δ6) ppm: 11.66 (m, 1H), 8.47-8.33 (dd, 1, J=5.86 Hz, J=2.20 Hz), 8.28-8.09 (m, 2H), 7.46-7.30 (m, 21H), 6.99-6.82 (m, 1H), 5.04-4.75 (m, 4H), 4.57-4.36 (m, 2H), 3.34 (s, 3H), 1.48-1.32 (m, 3H);
1-ethoxycarbonyl-3-(3-phenoxy-benzamido)-5-[(2-methylthio)pyrimidin-4-yl]-4,6-dihydropyrrolo[3,4-c]pyrazole (80% yield)

1H-NMR (400 M DMSO-δ6) ppm: 11.68-11.53 (m, 1H), 8.15-8.05 (m, 1H), 7.91-7.85 (d, 1H, J=7.92 Hz), 7.73-7.64 (m, 1H), 7.60-7.53 (m, 1H), 7.49-7.43 (m, 2H), 7.34-7.26 (m, 1H), 7.26-7.18 (m, 1H), 7.12-7.06 (m, 2H), 6.41-6.31 (n, 1H), 4.96-4.62 (m, 4H), 4.49-4.07 (q, 2H, J=7.07 Hz), 2.48 (s, 3H), 1.45-1.33 (t, 3H, J=7.07 Hz);
1-ethoxycarbonyl-3-(2-naphthylacetamido)-5-[(2-methylthio)pyrimidin-4-yl]-4,6-dihydropyrrolo[3,4-c]pyrazole

1H-NMR (400 MHz, DMSO-δ6) ppm: 11.55-11.28 (m, 1H), 8.10-7.96 (m, 1H), 7.91-7.85 (m, 3H), 7.84-7.79 (m, 1H), 7.54-7.43 (m, 3H), 6.39-6.18 (m, 1H), 4.85-4.46 (m, 4H), 4.44-4.34 (q, 2H, J=7.08 Hz), 3.83 (s, 2H), 2.43 (s, 3H), 1.40-1.28 (t, 3H).
3-[4-(morpholin-1-yl)-benzamido]-1-ethoxycarbonyl-5-t-butyloxycarbonyl-4,6-dihydropyrrolo[3,4-c]pyrazole

1H-NMR (400, DMSO-δ6) ppm: 11.16 (s, bs, 1H), 7.98-7.01 (m, 4H), 4.85-4.46 (m, 4H), 4.55-4.42 (m, 6H), 3.36-2.48 (m, 8H), 2.27 (s, 3H), 1.50 (s, 9H), 1.37 (t, 3H);
3-[4-(N-methyl-piperazin-1-yl)-benzamido]-1-ethoxycarbonyl-5-t-butyloxycarbonyl-4,6-dihydropyrrolo[3,4-c]pyrazole

1H-NMR (400 MHz, DMSO-δ6) ppm: 11.14 (s, bs, 1H), 7.95-6.95 (m, 4H), 4.50 (m, 4H), 4.38 (q, 3H), 3.72-3.70 (m, 4H), 3.28-3.23 (m, 4H), 1.44 (s, 9H), 1.32 (t, 3H).

### EXAMPLE 6

### Preparation of 3-cyclobutanoylamido-1-ethoxycarbonyl-5-[(2-chloro)pyrimidin-4-yl]-4,6-dihydropyrrolo[3,4-c]pyrazole

Cyclobutanoyl chloride (19 ml, 17.03 mmol) was added to a suspension of 3-amino-1-ethoxycarbonyl-5-[(2-chloro)pyrimidin-4-yl]-4,6-dihydropyrrolo[3,4-c]pyrazole dihydrochloride (3.5 g, 11.35 mmol) in dry THF (220 ml) and dry pyridine (75 ml). The mixture was stirred at room temperature for 24 hours. The solvents were evaporated and the residue was taken up in hot water (75 ml), tire for about 30 minutes, filtered, washed with diethyl ether and dried under vacuum at 30° C. The title compound was obtained as a beige powder (42 g, 95%).

1H-NMR (400 MHz, DMSO-δ6) ppm: 10.94 (s 1H), 8.15 (d, 2H), 6.67 (d, 1H), 4.87-4.58 (m, 4H), 4.42 (q, 2H), 3.34 (m, 1H), 2.43-1.71 (m, 6H), 1.37 (t, 3H).

By operating in an analogous way the following compounds were also obtained:
3-thien-2-yl)-carboxamido-1-ethoxycarbonyl-5-[(2-chloro)pyrimidin-4-yl]-4,6-dihydropyrrolo[3,4-c]pyrazole

1H-NMR (400, DMSO-δ6) ppm: 11.3 (s 1H), 8.15 (2, 1H), 8.13-7.22 (m, 3H), 6 (d, 1H), 4.8-4.6 (m, 4H), 4.4 (q, 2H), 1.2 (t, 3H);
3-(4-methoxybenzamido)-1-ethoxycarbonyl-5-[(2-chloro)pyrimidin-4-yl]-4,6-dihydropyrrolo[3,4]pyrazole

1H-NMR (400 MHz, DMSO-δ6) ppm: 1138 (s 1H), 8.07 (m, 3H), 7.0 (d, 2H), 6.61 (d, 1H), 4.8-4.66 (m, 4H), 4.4 (q, 2H), 1.17 (t, 3H).

### EXAMPLE 7

### 1-ethoxycarbonyl-3-(4-fluoro-benzamido)-5-[(2-methanesulfonyl)pyrimidin-4-yl]-4,6-dihydropyrrolo[3,4-c]pyrazole

m-Chloroperbenzoic acid (2 eq.) was added to a suspension of 1-ethoxycarbonyl-3-(4-fluoro-benzamido)-5-[(2-methylthio)pyrimidin-4-yl]-4,6-dihydropyrrolo[3,4-c]pyrazole (100 mg, 1 eq.) in dichloromethane (25 ml). The mixture was stirred for 1 hour at room temperature then washed wilt water, 10% sodium thiosulfate, brine, dried over sodium sulfate and evaporated under vacuum. The crude was chromatographed on silica gel, using dichloromethane:ethyl acetate as eluent, to yield the title compound as a colorless powder (55% yield).

1H-NMR (400 MHz, DMSO-δ6) ppm: 11.66 (m, 1H), 8.47-8.33 (dd, 1H, J=5.86 Hz, J=2.20 Hz), 8.28-8.09 (n, 2H), 7.46-7.30 (m, 2H), 6.99-6.82 (m, 1H), 5.04-4.75 (m, 4H), 4.57-4.36 (m, 2H), 3.34 (s, 3H), 1.48-1.32 (m, 3H).

### EXAMPLE 8

### Preparation of 3-(4-tort-butyl-benzamido)-5[(2-methylthio)pyrimidin-4-yl]-4,6-dihydropyrrolo[3,4-c]pyrazole

A solution of 3-(tert-butylbenzamido)-1-ethoxycarbonyl-5-[(2-methylthio)pyrimidin-4,6-dihydropyrrolo[3,4-c]pyrazole (3.7 mmol) in methanol (15 ml) and triethylamine (1 ml), was stirred at 40° C. for 4 hours. After evaporation of the solvents, the solid was washed with diethyl ether and dried so as to obtain the title compound (yield 100%)

1H-NMR (400 MHz, DMSO-δ6) ppm: 12.48 (m, 1H), 10.87 (m, 1H), 8.12-8.04 (m, 1H), 8.02-7.86 (m, 2H), 7.66-7.42 (m, 2H), 6.39-6.28 (m, 1H), 4.87-4.42 (m, 4H), 2.49 (s, 3H), 1.34 (s, 9H).

Operating in an analogous way, the following compounds were also obtained:
3-(4-fluoro-benzamido)-5-[(2-methylthio)pyridin-4-yl]-4,6-dihydropyrrolo[3,4-c]pyrazole

1H-NMR (400 MHz, DMSO-d6) ppm: 12.51 (m, 1H), 11.00 (m, 1H), 8.18-7.96 (m, 3H), 7.47-7.24 (m, 2H), 6.42-6.28 (m, 1H), 4.85-4.28 (m, 4H), 2.49 (s, 3H);
3-(3-phenoxy-benzamido)-5-[(2-methylthio)pyrimidin-4-yl]-4,6-dihydropyrrolo[3,4-c]pyrazole

1H-NMR (400 MHz, DMSO-δ6) ppm: 12.50 (m, 1H), 11.04 (m, 1H), 8.11-8.02 (m, 1H), 7.86-7.78 (m, 1H), 7.70-7.61 (m, 1H), 7.59-7.52 (m, 1H), 7.49-7.42 (m, 2H), 7.31-7.25 (m, 1H), 7.24-7.18 (t, 1H, J=7.31 Hz), 7.12-7.07 (m, 2H), 6.38-6.31 (m, 1H), 4.76-4.46 (m, 4H), 2.48 (s, 3H).

### EXAMPLE 9

### Preparation of 3-(4-tert-butyl-benzamido)-1-polystyrenetrityl-5-[(2-methylthio)pyrimidin-4-yl]-4,6-dihydropyrrolo[3,4-c]pyrazole

Trityl chloride resin (1.76 g, loading 1.27 mmol/g) was swelled with 10 ml of dichloromethane. A solution of 3-(tert-butylbenzamido)-5-[(2-methylthio)pyrimidin-4-yl]-4,6-dihydropyrrolo[3,4-c]pyrazole (1 g, 2.62 mmol) in 40 ml of dimethylformamide was added therein.

The mixture was stirred at room temperature for 48 hours. After filtration, the resin was washed with dichloromethane (2×20 ml), MeOH (2×20 ml), dimethylformamide (2×20 ml) dichloromethane (3×20 ml) and diethyl ether (2×20 ml). The resin supporting the title compound was dried under vacuum (loading 50%).

By operating in an analogous way, the following compounds may be also obtained:
3-(4-fluoro-benzamido)-1-polystyrenetrityl-5-[(2-methylthio)pyrimidin-4-yl]-4,6-dihydropyrrolo[3,4-c]pyrazole;
3-(3-phenoxy-benzamido)-1-polystyrenetrityl-5-[(2-methylthio)pyrimidin-4-yl]-4,6-dihydropyrrolo[3,4-c]pyrazole.

### EXAMPLE 10

### Preparation of 3-(4-tert-butyl-benzamido)-1-polystyrenetrityl-5-[(2-methanesulfonyl)pyrimidin-4-yl]-4,6]-dihydropyrrolo[3,4-c]pyrazole

A solution of m-chloroperbenzoic acid/NaOH (1/1) in dioxane, was added dropwise to 3-(4-tert-butyl-benzamido)-1-polystyrenetrityl-5-[(2-methylthio)pyrimidin-4-yl]-dihydropyrrolo[3,4-c]pyrazole. The mixture was stirred for 1 hour at room temperature. After filtration, the resin was washed with dichloromethane (2×20 ml), MeOH (2×20 ml), dimethylformamide (2×20 ml), dichloromethane (3×20 ml) and diethyl ether (2×20 ml) and dried under vacuum, yielding the title compound.

By operating in an analogous way, the following compounds may be also obtained:
3-(4-fluoro-benzamido)-1-polystyrenetrityl-5-[(2-methanesulfonyl)pyrimidin-4-yl]-4,6-dihydropyrrolo[3,4-c]pyrazole;
3-(3-phenoxy-benzamido)-1-polystyrenetrityl-5-[(2-methanesulfonyl)pyrimidin-4-yl]-4,6-dihydropyrrolo[3,4-c]pyrazol.

### EXAMPLE 11

### Preparation of 3-(4-tert-butyl-benzamido)-1-polystyrenetrityl-5[(2-propylamino)pyrimidin-4-yl]-4,6-dihydropyrrolo[3,4-c]pyrazole

Propylamine (30 eq.) was added to a suspension of 3-(4-tert-butyl-benzamido)-1-polystyrenetrityl-5-[(2-methanesulfonyl)pyrimidin-4-yl]-4,6-dihydropyrrolo[3,4-c]pyrazole (0.975 g, 0.57 mmol), in dioxane (3 ml). The mixture was stirred for 20 hours at 80° C. After filtration, the resin was washed with dichloromethane (2×20 ml), MeOH (2×20 ml), dimethylformamide (2×20 ml), dichloromethane (3×20 ml) and diethyl ether (2×20 ml). Then, the resin was dried under vacuum affording the title compound.

By operating in an analogous way and by using a suitable amine, the following compounds may be also obtained:
3-(4-fluoro-benzamido)-1-polystyrenetrityl-5-[(2-propylamino)pyrimidin-4-yl]-4,6-dihydropyrrolo[3,4-e]pyrazole;
3-(3-phenoxy-benzamido)-1-polystyrenetrityl-5-[(2-propylamino)pyrimidin-4-yl]-4,6-dihydropyrrolo[3,4-c]pyrazole;
3-(4-fluoro-benzamido)-1-polystyrenetrityl-5-[(2-benzylamino)pyrimidin-4-yl]-4,6-dihydropyrrolo[3,4-c]pyrazole;
3-(4-tert-butyl-benzamido)-1-polystyrenetrityl-5-[(2-benzylamino)pyrimidin-4-yl]-4,6-dihydropyrrolo[3,4-c]pyrazole;
3-(3-phenoxy-benzamido)-1-polystyrenetrityl-5-[(2-benzylamino)pyrimidin-4-yl]-4,6-dihydropyrrolo[3,4-c]pyrazole;
3-(4-fluoro-benzamido)-1-polystyrenetrityl-5-[2-(N-morpholino)pyrimidin-4-yl]-4,6-dihydropyrrolo[3,4]pyrazole;
3-(4-tert-butyl-benzamido)-1-polystyrenetrityl-5-[2-(N-morpholino)pyrimidin-4-yl]-4,6-dihydropyrrolo[3,4-c]pyrazole;
3-(3-phenoxy-benzamido)-1-polystyrenetrityl-5-[2-(N-morpholino)pyrimidin-4-yl]-4,6-dihydropyrrolo[3,4]pyrazole;
3-(4-fluoro-benzamido)-1-polystyrenetrityl-5-[2-(4-methyl-piperazino) pyrimidin-4-yl]-4,6-dihydropyrrolo[3,4-c]pyrazole;
3-(4-tert-butyl-benzamido)-1-polystyrenetrityl-5-[2 methyl-piperazino)pyrimidin-4-yl]-4,6-dihydropyrrolo[3,4-c]pyrazole;
3-(3-phenoxy-benzamido)-1-polystyrenetrityl-5-[2-(4-methyl-piperazino)pyrimidin-4-yl]-4,6-dihydropyrrolo[3,4-c]pyrazole;
3-(4-fluoro-benzamido)-1-polystyrenetrityl-5-[2-(4-methyl-piperidino) pyrimidin-4-yl]-4,6-dihydropyrrolo[3,4-c]pyrazole;
3-(4-tert-butyl-benzamido)-1-polystyrenetrityl-5-[2-(4-methyl-piperidino)pyrimidin-4-yl]-4,6-dihydropyrrolo[3,4-c]pyrazole;
3-(3-phenoxy-benzamido)-1-polystyrenetrityl-5-[2-(4-methyl-piperidino)pyrimidin-4-yl]-4,6-dihydropyrrolo[3,4-c]pyrazole;
3-(4-fluoro-benzamido)-1-polystyrenetrityl-5-[2-(4-dimethyl ethylendiamino) pyrimidin-4-yl]-4,6-dihydropyrrolo[3,4-c]pyrazole;
3-(4-tert-butyl-benzamido)-1-polystyrenetrityl-5-[2-(4-ethyl-ethylendiamino) pyrimidin-4-yl]-4,6-dihydropyrrolo[3,4-e]pyrazole;
3-(3-phenoxy-benzamido)-1-polystyrenetrityl-5-[2-(4-dimethyl-ethylendiamino) pyrimidin-4-yl]-4,6-dihydropyrrolo[3,4-c]pyrazole;
3-(4-fluoro-benzamido)-1-polystyrenetrityl-5-[2-(1,4-trimethyl-ethylendiamino) pyrimidin-4-yl]-4,6-dihydropyrrolo[3,4-c]pyrazole;
3-(4-tert-butyl-benzamido)-1-polystyrenetrityl-5-[2-(1,4-trimethyl-ethylendiamino) pyrimidin-4-yl]-4,6-dihydropyrrolo[3,4-c]pyrazole;
3-(3-phenoxy-benzamido)-1-polystyrenetrityl-5-[2-(1,4-trimethyl-ethylendiamino) pyrimidin-4-yl]-4,6-dihydropyrrolo[3,4-c]pyrazole;
3-(4-fluoro-benzamido)-1-polystyrenetrityl-5-[2-(4-hydroxymethyl piperidino) pyrimidin-4-yl]-4,6-dihydropyrrolo[3,4-c]pyrazole;
3-(4-tert-butyl-benzamido)-1-polystyrenetrityl-5-[2-(4-hydroxymethyl piperidino)pyrimidin-4-yl]-4,6-dihydropyrrolo[3,4-c]pyrazole;
3-(3-phenoxy-benzamido)-1-polystyrenetrityl-5-[2-(4-hydroxymethyl piperidino)pyrimidin-4-yl]-4,6-dihydropyrrolo[3,4-c]pyrazole,
3-(4-fluoro-benzamido)-1-polystyrenetrityl-5-{2-[4-(2-hydroxyethyl)piperazino]pyrimidin-4-yl}-4,6-dihydropyrrolo[3,4-c]pyrazole;
3-(4-tert-butyl-benzamido)-1-polystyrenetrityl-5-{2-[4-(2-hydroxyethyl)piperazino]pyrimidin-4-yl}-4,6-dihydropyrrolo[3,4-c]pyrazole;
3-(3-phenoxy-benzamido)-1-polystyrenetrityl-5-{2-[4-(2-hydroxyethyl)piperazino]pyrimidin-4-yl}-4,6-dihydropyrrolo[3,4-c]pyrazole;
3-(4-fluoro-benzamido)-1-polystyrenetrityl-5-[(2-cyclopropylamino) pyrimidin-4-yl]-4,6-dihydropyrrolo[3,4-c]pyrazole;
3-(4-tert-butyl-benzamido)-1-polystyrenetrityl-5-[(2-cyclopropylamino)pyrimidin-4-yl]-4,6-dihydropyrrolo[3,4-c]pyrazole;
3-(3-phenoxy-benzamido)-1-polystyrenetrityl-5-[(2-cyclopropylamino)pyrimidin-4-yl]-4,6-dihydropyrrolo[3,4-c]pyrazole.

### EXAMPLE 12

### Preparation of 3-(4-tert-butyl-benzamido)-1-polystyrenetrityl-5-[(2-phenylamino)pyrimidin-4-yl]-4,6-dihydropyrrolo[3,4-c]pyrazole

A suspension of 3-(4-tert-butyl-benzamido)-1-polystyrenetrityl-5-[(2-methanesulfonyl)pyrimidin-4-yl]-4,6-dihydropyrrolo[3,4-c]pyrazole in neat aniline was placed in a microwave oven for 20 minutes at 140° C.

After filtration, the resin was washed with dichloromethane (2×20 ml), MeOH (2×20 ml), dimethylformamide (2×20 ml) dichloromethane (3×20 ml) and diethyl ether (2×20 ml). The resin was dried under vacuum affording the title compound.

By operating in an analogous way, the following compounds may be also obtained:
3-(4-fluoro-benzamido)-1-polystyrenetrityl-5-[(2-phenylamino) pyrimidin-4-yl]-4,6-dihydropyrrolo[3,4-c]pyrazole;
3-(3-phenoxy-benzamido)-1-polystyrenetrityl-5-[(2-phenylamino)pyrimidin-4-yl]-4,6-dihydropyrrolo[3,4-c]pyrazole.

### EXAMPLE 13

### Preparation of 3-(4-tert-butyl-benzamido)-1-polystyrenetrityl-5-[(2-ethyloxy)pyrimidin-4-yl]-4,6-dihydropyrrolo[3,4-e]pyrazole

Sodium ethylate (30 eq) was added to a suspension of 3-(4-tert-butyl-benzamido)-1-polystyrenetrityl-5-[(2-methanesulfonyl)pyrimidin-4-yl]-4,6-dihydropyrrolo[3,4-c]pyrazole (0.975 g, 0.57 mmol) in dioxane (3 ml). The mixture was stir for 20 hours at 80° C. After filtration, the resin was washed with dichloromethane (2×20 ml), MeOH (2×20 ml), dimethylformamide (2×20 ml), dichloromethane (3×20 ml) and diethyl ether (2×20 ml). The resin was dried under vacuum affording the tile compound (yield 30%).

By operating in an analogous way and by using a suitable alcoholate, the following compounds may be also obtained:
3-(4-fluoro-benzamido)-1-polystyrenetrityl-5-[(2-ethyloxy) pyrimidin-4-yl]-4,6-dihydropyrrolo[3,4-c]pyrazole;
3-(3-phenoxy-benzamido)-1-polystyrenetrityl-5-[(2-ethyloxy)pyrimidin-4-yl]-4,6-dihydropyrrolo[3,4-c]pyrazole;
3-(4-fluoro-benzamido)-1-polystyrenetrityl-5-[2-(1-methylpiperidin-4-yloxy) pyrimidin-4-yl]-4,6-dihydropyrrolo[3,4-c]pyrazole;
3-(4-tert-butyl-benzamido)-1-polystyrenetrityl-5-[2-(1-methylpiperidin-4-yloxy) pyrimidin-4-yl]-4,6-dihydropyrrolo[3,4-c]pyrazole;
3-(3-phenoxy-benzamido)-1-polystyrenetrityl-5-[2-(1-methylpiperidin-4-yloxy)pyrimidin-4-yl]-4,6-dihydropyrrolo[3,4-c]pyrazole.

### EXAMPLE 14

### Preparation of 3-(4-tert-butyl-benzamido)-5-[(2-propylamino)pyrimidin-4-yl]-4,6-dihydropyrrolo[3,4-c]pyrazole

A solution of trifluoro acetic acid in dichloromethane (20/80) was added to the resin 3-(4-tert-butyl-benzamido)-1-polystyrenetrityl-5-[(2-propylamino)pyrimidin-4-yl]-4, dihydropyrrolo[3,4-c]pyrazole. The suspension was stirred for 15 minutes at room temperature.

After filtration, the solvent was evaporated and the crude was neutralized with NH₄OH and dried under vacuum thus affording the title compound (yield 76%).

1H-NMR (400 M DMSO-δ6) ppm: 12.38 (m, 1H), 10.84 (m, 1H), 7.99-7.89 (d, 2H, J=7.93 Hz), 7.81-7.78 (d, 1H, J=5.85 Hz), 7.56-7.48 (d, 2H, J=7.93 Hz), 6.69-6.42 (m, 1H), 5.81-5.75 (d, 1H, 3=5.85 Hz), 4.75-4.30 (m, 4H), 3.23-3.14 (m, 2H), 1.60-1.44 (m, 2H), 1.30 (s, 9M), 0.90-0.83 (t, 3H, J=7.44 Hz).

By operating in an analogous way, the following compounds were also obtained:
3-(4-fluoro-benzamido)-5-[(2-propylamino)pyrimidin-4-yl]-4,6-dihydropyrrolo[3,4-c]pyrazole (yield 90%)

1H-NMR (400 MHz, DMSO-δ6) ppm: 12.46 (m, 1H), 10.97 (m, 1H), 8.21-7.94 (m, 2H), 7.89-7.71 (d, 1H, J=5.85 Hz), 7.49-7.24 (m, 2H), 6.83-6.49 (m, 1H), 5.94-5.76 (d, 1H, J=5.61 Hz), 4.86-4.32 (m, 4H), 3.47-3.05 (m, 2H), 1.66-1.42 (m, 2H), 1.00-0.73 (t, 3H, J=7.43 Hz);
3-(3-phenoxy-benzamido)-5-[(2-propylamino)pyrimidin-4-yl]-4,6-dihydropyrrolo[3,4-c]pyrazole (yield 67%)

1H-NMR (400 MHz, DMSO-δ6) ppm: 12.45 (m, 1H), 10.98 (m, 1H), 7.92-7.05 (m, 10H), 6.76-6.34 (m, 1H), 5.92-5.72 (d, 1H, J=5.86 Hz), 4.87-4.28 (m, 4H), 3.45-3.05 (m, 2H), 1.65-1.40 (m, 2H), 1.00-0.78 (t, 3H, J=7.32 Hz);
3-(4-tert-butyl-benzamido)-5-[(2-benzylamino)pyrimidin-4-yl]-4,6-dihydropyrrolo[3,4-c]pyrazole (yield 35%)

1H-NMR (400 M DMSO-δ6) ppm: 12.41 (n, 1H), 10.83 (m, 1H), 8.10-7.89 (m, 2H), 7.86-7.78 (d, 1H, J=5.85 Hz), 7.64-7.08 (m, 8H), 5.95-5.76 (m, 1H), 4.87-4.28 (m, 6H), 1.33 (s, 9H);
3-(4-fluoro-benzamido)-5-[(2-benzylamino)pyrimidin-4-yl]-4,6-dihydropyrrolo[3,4-c]pyrazole (yield 90%)

1H-NMR (400 MHz, DMSO-66) ppm: 12.45 (m, 1H), 10.96 (m, 1H), 8.24-7.96 (m, 2H), 7.89-7.77 (d, 1H, J=5.85 Hz), 7.49-7.02 (m, 9H), 5.96-5.74 (m, 1H), 4.81-4.31 (m, 6H);
3-(3-phenoxy-benzamido)-5-[(2-benzylamino)pyrimidin-4-yl]-4,6-dihydropyrrolo[3,4-c]pyrazole (yield 36%)

1H-NMR (400 MHz, DMSO-δ6) ppm: 12.44 (m, 1H), 10.98 (m, 1M, 7.96-6.98 (m, 16H), 5.95.5.77 (m, 1H), 4.84-4.29 (m, 6H);
3-(4-tert-butyl-benzamido)-5-[2-morpholino)pyrimidin-4-yl]-4,6-dihydropyrrolo[3,4-c]pyrazole (yield 48%)

1H-NMR (400 MHz, DMSO-δ6) ppm: 12.45 (m, 1H), 10.87 (m, 1), 8.21-7.84 (m, 3H), 7.65-7.42 (m, 2H), 6.04-5.79 (d, 1H, J=5.68 Hz), 4.90-4.25 (m, 4H), 3.84-3.57 (m, 8H), 1.34 (s, 9H);
3-(3-phenoxy-benzamido)-5-[2-(N-morpholino)pyrimidin-4-yl]-4,6-dihydropyrrolo[3,4-c]pyrazole

1H-NMR (400 MHz, DMSO-δ6) ppm: 12.44 (m, 1H), 10.96 (m, 1H), 8.18-7.99 (m, 2H), 7.94-7.87 (m, 1H), 7.53-721 (m, 2H), 5.97-5.84 (m, 1H), 4.83-432 (m, 4H), 3.69-3.56 (m, 8H);
3-(3-phenoxy-benzamido)-5-[2-(N-morpholino)pyrimidin-4-yl]-4,6-dihydropyrrolo[3,4-c]pyrazole (yield 57%)

1H-NMR (400 MHz, DMSO-δ6) ppm: 12.47 (m, 1H), 11.00 (m, 1H), 7.98-7.89 (d, 1H, J=5.73 Hz), 7.87-7.18 (m, 8H), 7.14-7.00 (d, 2H, J=7.93 Hz), 6.02-5.84 (d, 1H, J=5.73 Hz), 4.78-4.38 (m, 4H), 3.75-3.58 (m, 8H);
3-(4-fluoro-benzamido)-5-[2-(4-methyl-piperidino)pyrimidin-4-yl]-4,6-dihydropyrrolo[3,4-c]pyrazole;
3-(4-tert-butyl-benzamido)-5-[2-(4-methyl-piperidino)pyrimidin-4-yl]-4,6-dihydropyrrolo[3,4-c]pyrazole;
3-(3-phenoxy-benzamido)-5-[2-(4-methyl-piperidino)pyrimidin-4-yl]-4,6-dihydropyrrolo[3,4-c]pyrazole;
3-(4-fluoro-benzamido)-5-[2-(4-methyl-piperazino)pyrimidin-4-yl-4,6-dihydropyrrolo[3,4-c]pyrazole;
3-(4-tert-butyl-benzamido)-5-[2-(4-methyl-piperazino)pyrimidin-4-yl]-4,6-dihydropyrrolo[3,4-c]pyrazole;
3-(3-phenoxy-benzamido)-5-[2-(4-methyl-piperazino)pyrimidin-4-yl]-4,6-dihydropyrrolo[3,4-c]pyrazole;
3-(4-fluoro-benzamido)-5-[2-(4-dimethyl ethylendiamino)pyrimidin-4-yl]-4,6-dihydropyrrolo[3,4-c]pyrazole;
3-(4-tert-butyl-benzamido)-5-[2-(4-dimethyl ethylendiamino)pyrimidin-4-yl]-4,6-dihydropyrrolo[3,4-c]pyrazole;
3-(3-phenoxy-benzamido)-5-[2-(4-dimethyl ethylendiamino)pyrimidin-4-yl]-4,6-dihydropyrrolo[3,4-c]pyrazole;
3-(4-fluoro-benzamido)-5-[2-(1,4-trimethyl ethylendiamino)pyrimidin-4-yl]-4,6-dihydropyrrolo[3,4-c]pyrazole;
3-(4-tert-butyl-benzamido)-5-[2-(1,4-trimethyl ethylendiamino)pyrimidin-4-yl]-4,6-dihydropyrrolo[3,4-c]pyrazole;
3-(3-phenoxy-benzamido)-5-[2-(1,4-trimethyl ethylendiamino)pyrimidin-4-yl]-4,6-dihydropyrrolo[3,4-c]pyrazole;
3-(4-fluoro-benzamido)-5-[2-(4-hydroxymethyl piperidino)pyrimidin-4-yl]-4,6-dihydropyrrolo[3,4-c]pyrazole;
3-(4-tert-butyl-benzamido)-5-[2-(4-hydroxymethyl)piperazino]pyrimidin-4-yl]-4,6-dihydropyrrolo[3,4-c]pyrazole;
3-(3-phenoxy-benzamido)-5-[2-(4-hydroxymethyl piperidino)pyrimidin-4-yl]-4,6-dihydropyrrolo[3,4-c]pyrazole;
3-(4-fluoro-benzamido)-5-{2-[4-(2-hydroxyethyl)piperazino]pyrimidin-4-yl}-4,6-dihydropyrrolo[3,4-c]pyrazole;
3-(4-tert-butyl-benzamido)-5-{2-[4-(2-hydroxyethyl)piperazino]pyrimidin-4-yl}-4,6-dihydropyrrolo[3,4-c]pyrazole;
3-(3-phenoxy-benzamido)-5-{2-[4-(2-hydroxyethyl)piperazino]pyrimidin-4-yl}-4,6-dihydropyrrolo[3,4-c]pyrazole;
3-(4-fluoro-benzamido)-5-[(2-cyclopropylamino)pyrimidin-4-yl]-4,6-dihydropyrrolo[3,4-c]pyrazole;
3-(4-tert-butyl-benzamido)-5-[(2-cyclopropylamino)pyrimidin-4-yl]-4,6-dihydropyrrolo[3,4-c]pyrazole;
3-(3-phenoxy-benzamido)-5-[(2-cyclopropylamino)pyrimidin-4-yl]-4,6-dihydropyrrolo[3,4-c]pyrazole

1H-NMR (400 MHz, DMSO-δ6) ppm: 12.45 (m, 1H), 10.99 (m, H), 8.04-7.04 (m, 10H), 6.93-6.45 (m, 1H), 5.96-5.78 (m, 1H), 4.90-4.30 (m, 4N), 2.80-2.62 (m, 1H), 0.71-0.35 (m, 4H);
3-(4-tert-butyl-benzamido)-5-[(2-phenylamino)pyrimidin-4-yl]-4,6-dihydropyrrolo[3,4-c]pyrazole;
3-(4-fluoro-benzamido)-5-[(2-phenylamino)pyrimidin-4-yl]-4,6-dihydropyrrolo[3,4-c]pyrazole

1H-NMR (400 MHz, DMSO-δ6) ppm: 12.51(m, 1H), 10.99 (m, 1H), 9.09 (s, 1H), 8.19-8.08 (m, 2H), 8.04-8.00 (d, 1H, J=5.83 Hz), 7.89-7.78 (m, 2H), 7.45-7.31 (m, 2H), 7.30-7.20 (m, 2H), 6.95-6.85 (m, 1H), 6.12-5.99 (m, 1H), 4.96-4.38 (m, 4H);
3-(3-phenoxy-benzamido)-5-[(2-phenylamino)pyrimidin-4-yl]-4,6-dihydropyrrolo[3,4-c]pyrazole;
3-(4-fluoro-benzamido)-5-[(2-ethyloxy)pyrimidin-4-yl]-4,6-dihydropyrrolo[3,4-c]pyrazole;
3-(4-tert-butyl-benzamido)-5-[(2-ethyloxy)pyrimidin-4-yl]-4,6-dihydropyrrolo[3,4-c]pyrazole;
3-(3-phenoxy-benzamido)-5-[(2-ethyloxy)pyrimidin-4-yl]-4,6-dihydropyrrolo[3,4-c]pyrazole

1H-NMR (400 MHz, DMSO-δ6) ppm: 12.50 (m, 1H), 11.01 (m, 1H), 8.09-7.98 (m, 1H), 7.89-7.01 (m, 9H), 6.33-6.13 (m, 1H), 4.81-4.43 (m, 4H), 4.37-4.16 (m, 2H), 1.40-1.21 (t, 3H, J=7.07 Hz);
3-(4-fluoro-benzamido)-5-[2-(1-methylpiperidin-4-yloxy) pyrimidin-4-yl]-4,6-dihydropyrrolo[3,4-c]pyrazole:
3-(4-tert-butyl-benzamido)-5-[2-(1-methylpiperidin-4-yloxy) pyrimidin-4-yl]-4,6-dihydropyrrolo[3,4-c]pyrazole;
3-(3-phenoxy-benzamido)-5-[2-(1-methylpiperidin-4-yloxy)pyrimidin-4-yl]-4,6-dihydropyrrolo[3,4-c]pyrazole.

### EXAMPLE 15

### Preparation of 3-(thien-2-yl)-carboxamido-5-[2-(4-methyl-piperazin-4-yl)pyrimidin-4-yl]-4,6-dihydropyrrolo[3,4-c]pyrazole

A suspension of 3-(thien-2-yl)-carboxamido-5-[(2-chloro)-pyrimidin-4-yl]-4,6-dihydropyrrolo[3,4-c]pyrazole (0.25 g, 0.6 mmol), 4-methyl piperazine (0.5 ml, 4.78 mmol) in iso-propanol (4 ml) was placed in a microwave oven for 45 minutes at 160° C.

After evaporation of the solvents, the mixture was taken up in dichloromethane (50 ml), washed with water (2×100 ml), brine, dried over sodium sulfate, filtered and dried under vacuum. The crude material was purified by flash chromatography on silica gel, using dichloromethane:methanol:ammonium hydroxide 95:5:0.5, to yield the title compound (0.115 g, 50%) as a beige powder.

1H NMR (400 MHz, DMSO-δ6) ppm: 12.47 (s,bs,1H), 11.03 (s,bs,1H), 8.13 (m,1H), 7.91 (m,2H), 7.87 (m,1H), 7.22 (m,1H), 5.89 (m,1H), 4.63 (m,4H), 3.72 (m,4H), 2.28 (m,4H), 2.24 (s,3H).

[M+H]⁺ 411.16; r.t. 2.450 min

By operating in an analogous way and by using the suitable amine, the following compounds of Table I were prepared:

TABLE I

| | | | r.t. | |
|---|---|---|---|---|
| Entry | Compound name | [M + H]+ | (min) | 1H-NMR(400 MHz, DMSO-d⁶)ppm |
| 1 | 3-cyclobutanoyl-carboxamido-5-[2- | 383.22 | 2.150 | |
| | (4-methyl-piperazin-4-yl)pyrimidin- | | | |
| | 4-yl]-4,6-dihydropyrrolo[3,4- | | | |
| | c]pyrazole | | | |
| 2 | 3-cyclobutanoyl-carboxamido-5-[2- | 370.19 | 2.720 | |
| | (morpholin-4-yl)pyrimidin-4-yl]-4,6- | | | |
| | dihydropyrrolo[3,4-c]pyrazole | | | |
| 3 | 3-cyclobutanoyl-carboxamido-5[2- | 390.20 | 3.750 | |
| | phenylmethylamino-pyrimidin-4-yl]- | | | |
| | 4,6-dihydropyrrolo[3,4-c]pyrazole | | | |
| 4 | 3-cyclobutanoyl-carboxamido-5-[2- | 342.20 | 3.220 | |
| | n-propylamino-pyrimidin-4-yl]-4,6- | | | |
| | dihydropyrrolo[3,4-c]pyrazole | | | |
| 5 | 3-cyclobutanoyl-carboxamido-5-[2- | 340.18 | 2.750 | |
| | cyclopropylamino-pyrimidin-4-yl]- | | | |
| | 4,6-dihydropyrrolo[3,4-c]pyrazole | | | |
| 6 | 3-cyclobutanoyl-carboxamido-5-[2- | 340.18 | 3.424 | |
| | allylamino-pyrimidin-4-yl]-4,6- | | | |
| | dihydropyrrolo[3,4-c]pyrazole | | | |
| 7 | 3-(thien-2-yl)-carboxamido-5-[2- | 398.13 | 3.231 | 12.43(s, bs, 1H)11.02(s, bs, 1H)8.11 |
| | (morpholin-4-yl)pyrimidin-4-yl]-4,6- | | | (m, 1H)7.86(d, 1H)7.82(d, 1H)7.35- |
| | dihydropyrrolo[3,4-c]pyrazole | | | 7.22(m, 6H)5.84(d, 1H)4.47-4.71 |
| | | | | (m, 4H)3.63-3.74(m, 8H) |
| 8 | 3-(thien-2-yl)-carboxamido-5-[2- | 418.14 | 4.117 | 12.49(s, 1H)11.04(s, 1H)8.14(s, 1H) |
| | phenylmethylamino-pyrimidin-4-yl]- | | | 7.94(d, J=5.85Hz, 1H)7.86(d, |
| | 4,6-dihydropyrrolo[3,4-c]pyrazole | | | J=3.54Hz, 1H)7.17-7.30(m, 1H)5.93 |
| | | | | (d, J=5.73Hz, 1H)4.49(m, 6H) |
| 9 | 3-(thien-2-yl)-carboxamido-5-[2-n- | 370.14 | 3.545 | 12.46(s, bs, 1H)11.02(8, 1H)8.13(m, |
| | propylamino-pyrimidin-4-yl]-4,6- | | | 1H)7.86(m, 1H)7.84(d, 1H)7.22(m, 1H) |
| | dihydropyrrolo[3,4-c]pyrazole | | | 6.58(m, bs, 1H)5.83(d, 1H)4.53(m, 4H) |
| | | | | 3.19(m, 2H)1.54(m, 2H)0.9(t, 3H) |
| 10 | 3-(thien-2-yl)-carboxamido-5-[2- | 368.12 | 3.197 | |
| | cyclopropylamino-pyrimidin-4-yl]- | | | |
| | 4,6-dihydropyrrolo[3,4-c]pyrazole | | | |
| 11 | 3-(4-methoxybenzamido)-5-(2-(4- | 435.22 | 2.384 | |
| | methyl-piperazin-4-yl)pyrimidin-4- | | | |
| | yl]-4,6-dihydropyrrolo[3,4- | | | |
| | c]pyrazole | | | |
| 12 | 3-(4-methoxybenzamido)-5-[2- | 422.19 | 3.311 | 12.43(s, bs, 1H)10.77(s, bs, 1H)8.02 |
| | (morpholin-4-yl)pyrimidin-4-yl]-4,6- | | | (m, bs, 2H)7.93(d, 1H)7.05(m, bs, 2H) |
| | dihydropyrrolo[3,4-c]pyrazole | | | 5.93(d, 1H)4.64-4.56(m, 4H)3.86(s, |
| | | | | 3H)3.68(m, 8H) |
| 13 | 3-(4-methoxybenzamido)-5-[2- | 442.19 | 4.260 | 12.40(s, bs1H)10.75(s, bs, 1H)8.03(m, |
| | phenylmethylamino-pyrimidin-4-yl]- | | | bs, 2H)7.83(d, 1H)7.36-7.20(m, 5H) |
| | 4,6-dihydropyrrolo[3,4-c]pyrazole | | | 7.06(m, bs, 2H)5.86(d, 1H)4.50(m, 6H) |
| | | | | 3.85(s, 3H) |
| 14 | 3-(4-methoxybenzamido)-5-[2-n- | 394.19 | 3.729 | 12.40(s, bs1H)10.78(s, bs, 1H)8.02(m, |
| | propylamino-pyrimidin-4-yl]-4,6- | | | bs, 2H)7.83(d, 1H)7.06(m, bs, 2H)6.74 |
| | dihydropyrrolo[3,4-c]pyrazole | | | (m, bs, 1H)5.88(d, 1H)4.73-4.54(m, 4H) |
| | | | | 3.88(s, 3H)3.28(m, 3H)1.55(m, 2H) |
| | | | | 0.91(tt, 3H) |
| 15 | 3-(4-methoxybenzamido)-5-[2- | 392.18 | 3.372 | 12.42(s, bs1H)10.76(s, bs, 1H)8.02(m, |
| | cyclopropylamino-pyrimidin-4-yl]- | | | bs, 2H)7.87(d, 1H)7.06(m, bs, 2H)5.94 |
| | 4,6-dihydropyrrolo[3,4-c]pyrazole | | | (d, 1H)4.72-4.47(m, 5H)3.85(s, 3H) |
| | | | | 2.73(m, 1H)0.7-0.46(m, 4H) |
| 16 | 3-[4-(N-Methyl-piperazin-1-yl)- | 503.29 | 1.636 | |
| | benzamido]-5-[2-(4-methyl- | | | |
| | piperazin-4-yl)pyrimidin-4-yl]-4,6- | | | |
| | dihydropyrrolo[3,4-c]pyrazole | | | |
| 17 | 3-[4-(N-Methyl-piperazin-1-yl)- | 490.26 | 2.849 | |
| | benzamido]-5-[2-(morpholin-4- | | | |
| | yl)pyrimidin-4-yl]-4,6- | | | |
| | dihydropyrrolo[3,4-c]pyrazole | | | |
| 18 | 3-[4-(N-Methyl-piperazin-1-yl)- | 510.27 | 2.977 | |
| | benzamido]-5-[2- | | | |
| | phenylmethylamino-pyrimidin-4-yl]- | | | |
| | 4.6-dihydropyrrolo[3,4-c]pyrazole - | | | |
| 19 | 3-[4-(N-Methyl-piperazin-1-yl)- | 462.27 | 2.67 | |
| | benzamido]-5-[2-n-propylamino- | | | |
| | pyrimidin-4-yl]-4,6- | | | |
| | dihydropyrrolo[3,4-c]pyrazole | | | |
| 20 | 3-[4-(N-Methyl-piperazin-1-yl)- | 460.25 | 2.164 | |
| | benzamido]-5-[2-cyclopropylamino- | | | |
| | pyrimidin-4-yl]-4,6- | | | |
| | dihydropyrrolo[3,4-c]pyrazole | | | |
| 21 | 3-[4-(morpholin-1-yl)-benzamido]- | 490.25 | 3.333 | |
| | 5-[2-(4-methyl-piperazin-4- | | | |
| | yl)pyrimidin-4-yl)-4,6- | | | |
| | dihydropyrrolo[3,4-c]pyrazole | | | |
| 22 | 3-(4-(morpholin-1-yl)-benzamido]- | 477.23 | 3.339 | |
| | 5-[2-(morpholin-4-yl)pyrimidin-4-yl]- | | | |
| | 4,6-dihydropyrrolo[3,4-c]pyrazole | | | |
| 23 | 3-[4-(morpholin-1-yl)-benzamido]- | 497.23 | 4.332 | |
| | 5-[2-phenylmethylamino-pyrimidin- | | | |
| | 4-yl]-4,6-dihydropyrrolo[3,4- | | | |
| | c]pyrazole | | | |
| 24 | 3-[4-(morpholin-1-yl)-benzamido]- | 449.23 | 3.861 | |
| | 5-[2-n-propylamino-pyrimidin-4-yl]- | | | |
| | 4,6-dihydropyrrolo[3,4-c]pyrazole | | | |
| 25 | 3-[4-(morpholin-1-yl)-benzamido]- | 447.23 | 3.409 | |
| | 5-[2-cyclopropylamino-pyrimidin-4- | | | |
| | yl]-4,6-dihydropyrrolo[3,4- | | | |
| | c]pyrazole | | | |
| 26 | 3-[(4-fluoro)-benzamido]-5-[2-(4- | 423.20 | 3.458 | |
| | methyl-piperazin-4-yl)pyrimidin-4- | | | |
| | yl]-4,6-dihydropyrrolo[3,4- | | | |
| | c]pyrazole | | | |
| 27 | 3-[(4-fluoro)-benzamido]-5-{2-[N- | 425.21 | 3.191 | |
| | methyl-N-(N′,N′- | | | |
| | dimethylaminoethyl)- | | | |
| | amino]pyrimidin-4-yl}-4,6- | | | |
| | dihydropyrrolo[3,4-c]pyrazole | | | |
| 28 | 3-[(4-fluoro)-benzamido]-5-{2-[N- | 411.20 | 2.840 | |
| | (N′,N′-dimethylaminoethyl)- | | | |
| | amino]pyrimidin-4-yl]-4,6-dihydropyrrolo | | | |
| | [3,4-c]pyrazole | | | |
| 29 | 3-[(4-fluoro)-benzamido]-5-[2-(4- | 422.20 | 5.048 | |
| | methyl-piperidin-1-yl)pyrimidin-4- | | | |
| | yl]-4,6-dihydropyrrolo[3,4- | | | |
| | c]pyrazole | | | |
| 30 | 3-[(4-fluoro)-benzamido]-5-[2-(4- | 438.20 | 3.606 | |
| | hydroxymethyl-piperidin-1- | | | |
| | yl)pyrimidin-4-yl]-4,6- | | | |
| | dihydropyrrolo[3,4-c]pyrazole | | | |
| 31 | 3-[(4-fluoro)-benzamido]-5-[2-(4- | 453.21 | 3.354 | |
| | hydroxy-ethyl-piperazin-1- | | | |
| | yl)pyrimidin-4-yl]-4,6- | | | |
| | dihydropyrrolo[3,4-c]pyrazole | | | |

### EXAMPLE 16

### Preparation of 3-[(4-tert-butyl-benzamido)-5-pyrimidin-2-yl]-4,6-dihydropyrrolo[3,4-c]pyrazole

A solution of 3-(4-tert-butyl-benzamido)-1-ethoxycarbonyl-4,6-dihydropyrrolo[3,4-c]pyrazole (50 mg, 0.14 mmol) in DMF (3 ml), was treated with 2-chloro-pyrimidine (32 mg, 0.28 mmol) and K₂CO₃ (0.21 mmol). The reaction mixture was stirred for 8 hours at 50° C. Methanol (5 ml) was then added to the mixture and the solution was stirred for 1 hour at room temperature. The solvent was evaporated and the solution was washed with water and the organic product was extracted with ethyl acetate. The organic layers were combined, washed with brine, dried over sodium sulfate, filtered and dried under vacuum. The crude material was purified by flash chromatography on silica gel, using cyclohexane:ethyl acetate as eluent to yield the title compound as a colorless solid (yield 50%).

By operating in an analogous way, the following compound was also obtained:
3-[(2-naphthylacetamido)-5-pyrimidin-2-yl]-4,6-dihydropyrrolo[3,4-c]pyrazole

1H-NMR (400 MHz, DMSO-δ6) ppm: 12.55-11.93 (m, 1H), 10.72 (bs, 1H), 8.39-8.31 (d, 2H, J=4.88 Hz), 7.91-7.83 (m, 3H), 7.80 (s, 1H), 7.54-7.38 (m, 3H), 6.69-6.59 (t, 1H, J=4.88 Hz), 4.55 (m, 4M), 3.79 (s, 2H).

### EXAMPLE 17

### Preparation of 3-(4-fluoro-benzamido)-5-(thiazol-2yl)-4,6-dihydropyrrolo[3,4-c]pyrazole

2-Bromo-thiazole (0.28 ml, 3.12 mmol) was added dropwise to a suspension of 3-(4-fluoro-benzamido)-1-ethoxycarbonyl-4,6-dihydropyrrolo[3,4-c]pyrazole (2.4 mmol) and K₂CO₃ (828 mg, 6 mmol), in DMSO (3 ml).

The reaction mixture was stirred at 100° C. for 48 hours. The solution was washed with water and the organic product was extracted with ethyl acetate. The organic layers were combined, washed with brine, dried over sodium sulfate, filtered and dried under vacuum. The crude material was purified by flash chromatography on silica gel, using cyclohexane:ethyl acetate as eluent to yield the title compound.

By operating in an analogous way, the following compounds were also obtained:
3-(4-fluoro-benzamido)-5-(benzothiazol-2yl)-4,6-dihydropyrrolo[3,4-c]pyrazole;
3-(4-fluoro-benzamido)-5-[6-chloro-(pyrimidin-4-yl)]-4,6-dihydropyrrolo[3,4-c]pyrazole

1H-NMR (400 MHz, DMSO-δ6) ppm: 12.54 (m, 1H), 11.01 (m, 1H), 8.41 (s, 1H), 8.27-7.90 (m, 2H), 7.61-7.14 (m, 2H), 6.83-6.57 (m, 1H), 5.04-4.29 (m, 4H).

### EXAMPLE 18

### Preparation of 3-amino-5-tert-butyloxycarbonyl-1-ethoxycarbonyl-pyrazolo[4,3-c]4,5,6,7-tetrahydropyridine

To a solution of 3-amino-5-tert-butyloxycarbonyl-pyrazolo[4,3-c]4,5,6,7-tetrahydro pyridine (94.1 g, 0.395 moles) and N,N-diisopropylethylamine (135 ml, 0.79 moles) in tetrahydrofuran (1700 ml), ethylchloroformate (98% assay, 37.6 ml, 0.395 moles) in tetrahydrofuran (300 ml) was added dropwise at 0° C. in 75 minutes. The mixture was stirred at 0° C. for 30 minutes then evaporated under vacuum. The residue was taken-up with water (1000 ml) and extracted with ethyl acetate (3×800 ml). The combined organic extracts were washed with brine (500 ml), dried over sodium sulfate, and evaporated to dryness. The crude mixture (144.5 g) was purified by flash-chromatography on silica gel employing CH₂Cl₂/EtOAc as eluent 87:13 to 1:1, to give the title compound as a colorless solid (29 g, yield 24%, HPLC Λ %=98.6) and the regioisomer 3-amino-5-tert-butyloxycarbonyl-2-ethoxycarbonyl-pyrazolo[4,3-c]4,5,6,7-tetrahydropyridine (62 g, yield 50%, HPLC Λ %=99).

1H-NMR (400 MHz, DMSO-δ6) ppm: 10.06 (s, broad signal, 2M), 4.3-4.2 (m, 4M), 3.6 (m, 2H), 2.6 (m, 2H), 1.27 (t, 3H) 1 (2, 9H).

### EXAMPLE 19

### Preparation of 3-amino-1-ethoxycarbonyl-4,6-pyrazolo[4,3-c]4,5,6,7-tetrahydropyridine

A solution of HCl 4M in dioxane (23 ml) was added to a solution of 3-amino-5-tert-butyloxycarbonyl-1-ethoxycarbonyl-pyrazolo[4,3-c]4,5,6,7-tetrahydropyridine (3.5 g, 0.011 mol); the white suspension was stirred at room temperature for 8 hours. The solid was filtered and washed with Et₂O. The title compound was obtained as a colorless powder (2.7 g, yield 100%) and was used without any further purification.

### EXAMPLE 20

### Preparation of 3-amino-1-ethoxycarbonyl-5-[(2-methylthio)pyrimidin-4-yl]-4,6-pyrazolo[4,3-c]4,5,6,7-tetrahydropyridine

2-Methylthio-4-chloropyrimidine (1.54 ml, 0.013 mol) and K₂CO₃ (3.3 g, 0.024 mol) were added to a solution of 3-amino-1-ethoxycarbonyl-4,6-pyrazolo[4,3-c]4,5,6,7-tetrahydro pyridine in DMSO (50 ml). The mixture was heated at 90° C. for 4 hours. Water (30 ml) was added and the mixture was extracted with ethyl acetate (2×20 ml). The organic layers were combined, washed with brine, dried over sodium sulfate, filtered and dried under vacuum. The crude material was purified by flash chromatography on silica gel, using cyclohexane:ethyl acetate as eluent to yield the title compound as a colorless solid (2.7 g, yield 82%).

1H-NMR (400 MHz, DMSO-δ6) ppm: 8.19-7.98 (m, 1H), 6.77-6.46 (m, 1H), 5.83-5.18 (m, 2H), 4.68-4.20 (m, 4H), 4.04-3.74 (m, 2H), 3.04-2.86 (m, 2H), 2.47 (s, 3H), 1.32-1.27 (t, 3H, J=7.07 Hz).

### EXAMPLE 21

### Preparation of 3-(tert-butyl-benzamido)-1-ethoxycarbonyl-5-[(2-methylthio)pyrimidin-4-yl]-4,6-pyrazolo[4,3-c]4,5,6,7-tetrahydro pyridine

Tri-(2-aminoethyl)-amine polystirene (4 g, 13 mmol) was added to a solution of 3-amino-1-ethoxycarbonyl-5-[(2-methylthio)pyrimidin-4-yl]-4,6-pyrazolo[4,3-c]4,5,6,7-tetrahydropyridine (860 mg, 2.6 mmol) in dry THF (30 ml). 4-Tert-butyl benzoyl chloride (0.85 ml, 4.16 mmol) was added dropwise to the suspension. The mixture was stirred at room temperature for 8 hours. The suspension was filtered, then water was added and the mixture was extracted with ethyl acetate. The organic layers were combined, washed with brine, dried over sodium sulfate, filtered and dried under vacuum. The crude material was purified by flash chromatography on silica gel, using cyclohexane:ethyl acetate as eluent to yield the title compound as a yellow oil (700 mg, 55%).

By operating in an analogous way the following compounds were also obtained using the suitable acyl chloride derivative:
3-(4-fluorobenzamido)-1-ethoxycarbonyl-5-[(2-methylthio)pyrimidin-4-yl]-4,6-pyrazolo[4,3-c]4,5,6,7-tetrahydro pyridine (yield 68%);
3-(3-phenoxy-benzamido)-1-ethoxycarbonyl-5-[(2-methylthio)pyrimidin-4-yl]-4,6-pyrazolo[4,3-c]4,5,6,7-tetrahydro pyridine (yield 72%);

### EXAMPLE 22

### Preparation of 3-(tert-butyl-benzamido)-5-[(2-methylthio)pyrimidin-4-yl]-4,6-pyrazolo[4,3-c]4,5,6,7-tetrahydropyridine

A solution of 3-(tert-butyl-benzamido)-1-ethoxycarbonyl-5-[(2-methylthio)pyridin-4-yl]-4,6-pyrazolo[4,3-c]-4,5,6,7-tetrahydropyridine (700 mg, 1.42 mmol) in MeOH (15 ml) and triethylamine (1 ml), was sired at room temperature for 4 hours. After evaporation of the solvent, the solid was washed with diethyl ether and dried, affording the title compound (600 mg, yield 100%).

1H-NMR (400 MHz, DMSO-δ6) ppm: 12.34(m, 1H), 10.43(m,1H), 8.08-8.01 (d, 1H J=6.10 Hz), 8.00-7.92 (d,2H, J=8.42 Hz), 7.61-7.46 (m, 2H), 6.68-6.44 (m, 1H), 4.73-4.42 (m, 2H), 4.15-3.77 (m, 2H), 2.85-2.70 (m, 2H), 2.44 (s, 3H), 1.34 (s, 9H).

By operating in an analogous way, the following compounds were also obtained:
3-(4-fluorobenzamido)-5-[(2-methylthio)pyrimidin-4-yl]-4,6-pyrazolo[4,3-c]4,5,6,7-tetrahydropyridine

1H-NMR (400 MHz, DMSO-δ6) ppm: 12.36(m, 1H), 10.55(m, 1H), 8.15-8.07 (m, 2H), 8.07-8.02 (d, 1H, =6.101 Hz), 7.41-7.32 (m, 2H), 6.65-6.56 (d, MHz, J=6.10 Hz), 4.64-4.47 (m, 2H), 4.08-3.91 (m, 2H), 2.83-2.72 (m, 2H), 2.44 (s, 3H);
3-(3-phenoxy-benzamido)-5-[(2-methylthio)pyrimidin-4-yl]-4,6-pyrazolo[43-c]4,5,6,7-tetrahydropyridine

1H-NMR (400 MHz, DMSO-δ6) ppm: 12.35(m, 1H), 10.56(m,1H), 8.06-8.01 (d, 1H, 1=6.22 Hz), 7.86-7.79 (d, 1H, J=8.05 Hz), 7.64 (s, 1H), 7.58-7.52 (t, 1H, J=8.05 Hz), 7.49-7.41 (n, 2H), 7.28-7.18 (m, 2H), 7.13-7.06 (m, 2H), 6.65-6.53 (d, 1H, J=6.20 Hz), 4.61-4.46 (m, 2H), 4.03-3.90 (m, 2H), 2.82-2.71 (m, 2H), 2.42 (s, 3H).

### EXAMPLE 23

### Preparation of 3-(4-tert-butyl-benzamido)-1-polystyrenetrityl-5-[(2-methylthio)pyrimidin-4-yl]-pyrazolo[4,3-c]4,5,6,7-tetrahydropyridine

Trityl chloride resin (1 g, loading 1.27 mmol/g) was swelled with 3 ml of dichloromethane. A solution of 3-(tert-butyl-benzamido)-5-[(2-methylthio)pyrimidin-4-yl]-4,6-pyrazolo[4,3-c]4,5,6,7-tetrahydropyridine (530 mg, 1.25 mmol) in 10 ml of dimethylformamide was added.

The mixture was stirred at room temperature for 24 hours. After filtration, the resin was washed with dichloromethane (2×20 ml), MeOH (2×20 ml), dimethylformamide (2×20 ml), dichloromethane (3×20 ml) and diethyl ether (2×20 ml).

The resin was dried under vacuum yielding the title compound (1.21 g, loading 50%).

By operating in an analogous way, the following compounds were also obtained:
3-(4-fluoro-benzamido)-1-polystyrenetrityl-5-[(2-methylthio)pyrimidin-4-yl]-pyrazolo[4,3-c]4,5,6,7-tetrahydro pyridine (loading 73%);
3-(3-phenoxy-benzamido)-1-polystyrenetrityl-5-[(2-methylthio)pyrimidin-4-yl]-pyrazolo[4,3-c]4,5,6,7-tetrahydro pyridine (loading 42%).

### EXAMPLE 24

### Preparation of 3-(4-tert-butyl-benzamido)-1-polystyrenetrityl-5-[(2-methanesulfonyl)pyrimidin-4-yl]-pyrazolo[4,3-c]4,5,6,7-tetrahydropyridine

A solution of m-chloroperbenzoic acid and NaOH (1/1) was added dropwise to a solution of 3-(4-tert-butyl-benzamido)-1-polystyrenetrityl-5-[(2-methylthio)pyrimidin-4-yl]-pyrazolo[4,3-c]4,5,6,7-tetrahydropyridine in dioxane. The mixture was stirred for 1 hour at room temperature. After filtration, the resin was washed with dichloromethane (2×20 ml), MeOH (2×20 ml), dimethylformamide (2×20 ml) dichloromethane (3×20 ml) and diethyl ether (2×20 ml) and was dried under vacuum yielding the title compound.

By operating in an analogous way, the following compounds may be also obtained:
3-(4-fluoro-benzamido)-1-polystyrenetrityl-5-[(2-methanesulfonyl)pyrimidin-4-yl]-pyrazolo[4,3-c]4,5,6,7-tetrahydropyridine;
3-(3-phenoxy-benzamido)-1-polystyrenetrityl-5-[(2-methanesulfonyl)pyrimidin-4-yl]-pyrazolo[4,3-c]4,5,6,7-tetrahydropyridine.

### EXAMPLE 25

### Preparation of 3-(4-tert-butyl-benzamido)-1-polystyrenetrityl-[(2-propylamino)pyrimidin-4-yl]-pyrazolo[4,3-c]4,5,6,7-tetrahydropyridine

Propylamine (30 eq) was added to a suspension of 3-(4-tert-butyl-benzamido)-1-polystyrenetrityl-5-[(2-methanesulfonyl)pyrimidin-4-yl]-pyrazolo[4,3-c]4,5,6,7-tetrahydropyridine in dioxane. The mixture was stirred for 20 hours at 80° C. After filtration, the resin was washed with dichloromethane (2×20 ml), MeOH (2×20 ml), dimethylformamide (2×20 ml) dichloromethane (3×20 ml) and diethyl ether (2×20 ml). The resin was dried under vacuum affording the title compound.

By operating in an analogous way, the following compounds may be also obtained:
3-(4-tert-butyl-benzamido)-1-polystyrenetrityl-5-[(2-benzylamino)pyrimidin-4-yl]-pyrazolo[4,3-c]4,5,6,7-tetrahydropyridine;
3-(4-tert-butyl-benzamido)-1-polystyrenetrityl-5-[(2-N-morpholino)pyrimidin-4-yl]-pyrazolo[4,3-c]4,5,6,7-tetrahydropyridine;
3-(4-fluoro-benzamido)-1-polystyrenetrityl-5-[(2-propylamino)pyrimidin-4-yl]-pyrazolo[4,3-c]4,5,6,7-tetrahydropyridine;
3-(3-phenoxy-benzamido)-1-polystyrenetrityl-5-[(2-propylamino)pyrimidin-4-yl]-pyrazolo[4,3-c]4,5,6,7-tetrahydropyridine;
3-(4-fluoro-benzamido)-1-polystyrenetrityl-5-[(2-benzylamino)pyrimidin-4-yl]-pyrazolo[4,3-c]4,5,6,7-tetrahydropyridine;
3-(3-phenoxy-benzamido)-1-polystyrenetrityl-5-[(2-benzylamino)pyrimidin-4-yl]-pyrazolo[4,3-c]4,5,6,7-tetrahydropyridine;
3-(fluoro-benzamido)-1-polystyrenetrityl-5-[(2-N-morpholino)pyrimidin-4-yl]-pyrazolo[4,3-c]4,5,6,7-tetrahydropyridine;
3-(3-phenoxy-benzamido)-1-polystyrenetrityl-5-[(2-N-morpholino)pyrimidin-4-yl]-30 pyrazolo[4,3-c]4,5,6,7-tetrahydropyridine.

### EXAMPLE 26

### Preparation of 3-(tert-butyl-benzamido)-5-[(2-propylamino)pyrimidin-4-yl]-4,6-pyrazolo[4,3-c]4,5,6,7-tetrahydro pyridine

A solution of trifluoroacetic acid in dichloromethane (20/80) was added to the resin 3-(4-tert-butyl-benzamido)-1-polystyrenetrityl-5-[(2-propylamino)pyridin-4-yl]-pyrazolo[4,3-c]4,5,6,7-tetrahydropyridine. The suspension was stirred for 15 minutes at room temperature.

After filtration, the solution was neutralized with saturated NaHCO₃ and after addition of ethyl acetate the organic phase was separated and evaporated. The colorless solid was washed with diethyl ether and dried under vacuum, thus affording the title compound.

1H-NMR (400 MIT DMSO-δ6) ppm: 12.16(m, 1H), 10.44(m,1H), 8.02-7.93 (d, 2H, J=8.41 Hz), 7.83-7.76 (d, 1H, k=5.85 Hz), 7.58-7.51 (m, 2H), 6.56-6.39 (m, 1H), 6.08-6.01 (d, 1H, J=5.97 Hz), 4.53-4.44 (m, 2H), 3.94-3.81 (m, 2H), 3.20-3.09 (X, 2H), 2.79-2.69 (m, 2H), 1.54-1.40 (m, 2H), 1.34 (s, 9H), 0.90-0.76 (t, 3H, J=7.32 Hz).

By operating in an analogous way, the following compounds were also obtained:
3-(4-fluoro-benzamido)-5-[2-propylamino)pyrimidin-4-yl]-pyrazolo[4,3-c]4,5,6,7-tetrahydropyridine

1H-NMR (400 MHz, DMSO-δ6) ppm: 12.10 (m, 1H), 10.53 (bs, 1H), 8.20-8.03 (m, 2H), 7.90-7.69 (d, 1H, J=5.97 Hz), 7.45-7.24 (m, 2H), 6.50 (bs, 1H), 6.19-5.88 (d, 1H, J=5.97 Hz), 4.48 (bs, 2H), 4.08-3.73 (m, 2H), 3.30-2.96 (m, 2H), 2.87-2.62 (m, 2H), 1.65-1.37 (m, 2H), 1.01-0.64 (t, 3H, J=7.32 Hz);
3-(3-phenoxy-benzamido)-5-[(2-propylamino)pyrimidin-4-yl]-pyrazolo[4,3-c]4,5,6,7-tetrahydropyridine

1H-NMR (400 MHz, DMSO-6) ppm: 12.33(bs, 1H), 10.56(bs, 1H), 7.86-7.78 (m, 2H), 7.65 (s, 1H), 7.58-7.52 (m, 1H), 7.48-7.41 (m, 2H), 7.29-7.23 (m, 1H), 7.23-7.17 (m, 1H), 7.12-7.05 (m, 2H), 6.96-6.73 (m, 1H), 6.25-6.10 (d, 1H, J=5.98 Hz), 4.61-4.44 (m, 2H), 4.02-3.83 (m, 2H), 3.22-3.11 (m, 2H), 2.81-2.67 (m, 2H), 1.57-1.41 (m, 2H), 0.89-0.76 (t, 3H, J=7.08 Hz);
3-(tert-buty-benzamido)-5-[(2-morpholino)pyrimidin-4-yl]-4,6-pyrazolo[4,3-c]4,5,6,7-tetrahydropyridine;
3-(4-fluorobenzamido)-5-[(2-morpholino)pyrimidin-4-yl]-4,6-pyrazolo[4,3-c]4,5,6,7-tetrahydropyridine

1H-NMR (400 MHz, DMSO-δ6) ppm: 12.32(bs, 1H), 10.51(bs,1H), 8.14-8.06 (m, 2H), 7.93-7.88 (d, 1H, J=5.98 Hz), 7.40-7.32 (t, 2H, J=8.78 Hz), 6.27-6.06 (d, 1H, 3=5.85 Hz), 4.59-4.37 (m, 2H), 4.01-3.87 (m, 2H), 3.72-3.55 (m, 8H), 2.82-2.65 (m, 2H);
3-(3-phenoxy-benzamido)-5-[(2-morpholino)pyrimidin-4-yl]-4,6-pyrazolo[4,3-c]4,5,6,7-tetrahydropyridine

1H-NMR (400 MHz, DMSO-δ6) ppm: 12.32(bs, 1H), 10.53(bs,1H), 7.92-7.88 (d, 1H, J=6.09 Hz), 7.84-7.79 (d, 1H, J=7.68 Hz), 7.63 (bs, 1H), 7.58-7.52 (t, 1H, J=7.92 Hz), 7.48-7.41 (m, 2H), 7.28-7.17 (m, 2H), 7.13-7.06 (m, 2H), 6.31-6.04 (d, 1H, J=5.61 Hz), 4.58-4.39 (m, 2H), 4.01-3.86 (m, 2H), 3.70-3.56 (m, 8H), 2.80-2.66 (m, 2H);
3-(tert-butyl-benzamido)-5-[(2-benzylamino)pyrimidin-4-yl]-4,6-pyrazolo[4,3-c]4,5,6,7-tetrahydropyridine

1H-NMR (400 MHz, DMSO-6) ppm: 12.26(m, 1H), 10.42(bs,1H), 8.04-7.95 (d, 2, J=8.54 Hz), 7.82-7.76 (d, 1H, J=5.97 Hz), 7.57-7.50 (d, 2H, J=8.54 Hz), 7.32-7.03 (m, 6H), 6.14-6.99 (m, 1H), 4.53-4.36 (m, 4H), 3.92-3.78 (m, 2H), 2.77-2.58 (m, 2H), 1.33 (s, 9H);
3-(4-fluoro benzamido)-5-[(2-benzylamino)pyrimidin-4-yl]-4,6-pyrazolo[4,3-c]4,5,6,7-tetrahydropyridine

1H-NMR (400 MHz, DMSO-δ6) ppm: 12.29(bs, 1H), 10.54(bs,1H), 8.17-8.08 (m, 2H), 7.82-7.76 (d, 1H, 3=5.97 Hz), 7.40-7.32 (m, 2H), 7.31-7.06 (m, 6H), 6.14-6.02 (d, 1H, J=5.97 Hz), 4.52-4.35 (m, 4H), 3.94-3.76 (m, 2H), 2.77-2.59 (m, 2H);
3-(3-phenoxy-benzamido)-5-[(2-benzylamino)pyrimidin-4-yl]-4,6-pyrazolo[4,3-c]4,5,6,7-tetrahydropyridine

1H-NMR (400 MHz, DMSO-δ6) ppm: 12.31(bs, 1H), 10.57(bs,1H), 7.88-7.82 (m, 1H), 7.82-7.78 (d, 1H, J=6.22 Hz), 7.68 (bs, 1H), 7.59-7.51 (m, 1H), 7.47-7.39 (m, 2H), 7.34-7.13 (m, 71, 7.11-7.03 (d, 2H, J=7.80 Hz), 6.25-6.05 (d, 1H, J=6.22), 4.54-4.46 (m, 2H), 4.44-4.37 (d, 2H, 3=6.10 Hz), 3.93-3.81 (m, 2H), 2.74-2.62 (m, 2H);
1-ethoxycarbonyl-3-(4-fluoro-benzamide)-5-[(2-methanesulfonyl)pyrimidin-4-yl]-4,6-dihydropyrrolo[3,4-c]pyrazole

1H-NMR (400 MHz, DMSO-δ6) ppm: 11.66 (m, 1H), 8.47-8.33 (dd, 1H, J=5.86 Hz, J=2.20 Hz), 8.28-8.09 (m, 2H), 7.46-7.30 (m, 2H), 6.99-6.82 (m, 1H), 5.04-4.75 (m, 4H), 4.57-4.36 (m, 2H), 3.34 (s, 3H), 1.48-1.32 (m, 3H).

1. A method for treating diseases caused by and/or associated with an altered protein kinase activity which comprises administering to a mammal in need thereof an effective amount of a pyrazole represented by formula (I) 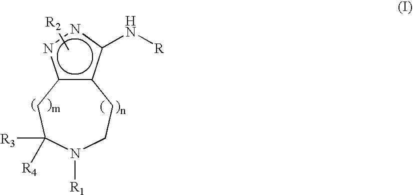
wherein
R is a hydrogen atom or a group selected from —COR′, —COOR′, —CONHR′, —C(═NH)NHR′, —SO₂R′ or —SO₂NR′R′;
R₁ is an optionally substituted 5 or 6 membered heterocyclic group with from 1 to 3 heteroatoms or heteroatomic groups selected from N, NR′, O or S, optionally benzocondensed;
R₂ is hydrogen or it is selected from the group consisting of R′, —COR′, —COOR′, —CONR′R″ or —S(O)_{q}R′;
R₃ and R₄ are both hydrogen atoms or methyl groups or, together with the carbon atom to which they are attached, form a cyclopropyl group;
R′ and R″ are, the same or different and independently in each of the above occasions, a hydrogen atom or an optionally substituted group selected from straight or branched C₁-C₆ alkyl, C₃-C₆ cycloalkyl, aryl, aryl C₁-C₆ alkyl heterocyclyl or heterocyclyl C₁-C₆ alkyl;
m and n are 0 or 1, provided that they are not both 1;
q is 0 or an integer from 1 to 2;
and the pharmaceutically acceptable salts thereof.

2. The method of claim 1 wherein the disease caused by and/or associated with an altered protein kinase activity is a cell proliferative disorder selected from the group consisting of cancer, Alzheimer's disease, viral infections, auto-immune diseases and neurodegenerative disorders.

3. The method of claim 2 wherein the cancer is selected from carcinoma, squamous cell carcinoma, hematopoietic tumors of lymphoid or myeloid lineage, tumors of mesenchymal origin, tumors of the central and peripheral nervous system, melanoma, seminoma, teratocarcinoma, osteosarcoma, xeroderma pigmentosum, keratoxanthoma, thyroid follicular cancer and Kaposi's sarcoma.

4. The method of claim 1 wherein the cell proliferative disorder is selected from benign prostate hyperplasia, familial adenomatosis, polyposis, neuro-fibromatosis, psoriasis, vascular smooth cell proliferation associated with atherosclerosis, pulmonary fibrosis, arthritis glomerulonephritis and post-surgical stenosis and restenosis.

5. The method of claim 1 which provides tumor angiogenesis and metastasis inhibition.

6. The method of claim 1 further comprising subjecting the mammal in need thereof to a radiation therapy or chemotherapy regimen in combination with at least one cytostatic or cytotoxic agent.

7. The method of claim 1 wherein the mammal in need thereof is a human.

8. A method for inhibiting protein kinase activity which comprises contacting the said kinase with an effective amount of a compound of formula (I) as defined in claim 1.

9. A pyrazole represented by formula (I) 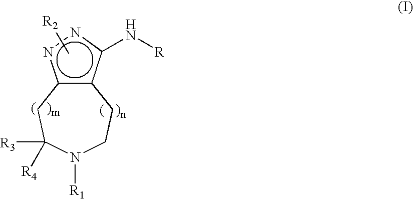
wherein
R is a hydrogen atom or a group selected from —COR′, —COOR′, —CONHR′, —C(═NH)NHR′, —SO₂R′ or —SO₂NR′R″;
R₁ is an optionally substituted 5 or 6 membered heterocyclic group with from 1 to 3 heteroatoms or heteroatomic groups selected from N, NR′, O or S, optionally benzocondensed;
R₂ is hydrogen or it is selected from the group consisting of R′, —COR′, —COOR′, —CONR′R″ or —S(O)_{q}R′;
R₃ and R₄ are both hydrogen atoms or methyl groups or, together with the carbon atom to which they are attached, form a cyclopropyl group;
R′ and R″ are, the same or different and independently in each of the above occasions, a hydrogen atom or an optionally substituted group selected from straight or branched C₁-C₆ alkyl, C₃-C₆ cycloalkyl, aryl, aryl C₁-C₆ alkyl, heterocyclyl or heterocyclyl C₁-C₆ alkyl;
m and n are 0 or 1, provided that they are not both 1;
q is 0 or an integer from 1 to 2;
and the pharmaceutically acceptable salts thereof.

10. A compound of formula (I) according to claim 9 wherein R₂ is hydrogen or a group —COOR′ and R′ is a straight or branched C₁-C₆ alkyl group.

11. A compound of formula (I) according to claim 10 wherein R′ is ethyl.

12. A compound of formula (I) according to claim 9 wherein R₁ is an optionally substituted heterocycle selected from pyrimidine, thiazole or benzothiazole.

13. A compound of formula (I) according to claim 12 wherein R₁ is a pyrimidine ring optionally substituted by one or more groups selected from halogen, heterocycles, alkylheterocycles, hydroxyalkylheterocycles, alkoxy, heterocyclyloxy, alkylheterocyclyloxy, alkylthio, alkylsulfonyl, alkylamino, cycloalkylamino, arylamino and arylalkylamino.

14. A compound of formula (I) according to claim 9 wherein m is 0 or 1 and n is 0.

15. A compound of formula (I) according to claim 9 wherein R₁, R′ and R″ are optionally substituted, in any of their free positions, by one or more groups selected from: halogen, nitro, oxo groups (═O), carboxy, cyano, alkyl polyfluorinated alkyl, hydroxyalkyl, alkenyl, alkynyl, cycloalkyl, aryl, heterocyclyl, amino groups and derivatives thereof such as aminoalkyl, alkylaminoalkyl, dialkylaminoalkyl, alkylamino, dialkylamino, cycloalkylamino, arylamino, diarylamino, arylalkylamino, ureido, alkylureido or arylureido; carbonylamino groups and derivatives thereof such as formylamino, alkylcarbonylamino, alkenylcarbonylamino, arylcarbonylamino, alkoxycarbonylamino; hydroxy groups and derivatives thereof such as alkoxy, aryloxy, arylalkyloxy, heterocyclyloxy, alkylcarbonyloxy, arylcarbonyloxy, cycloalkenyloxy or alkylideneaminooxy, carbonyl groups and derivatives thereof such as alkylcarbonyl, arylcarbonyl, alkoxycarbonyl, aryloxycarbonyl, cycloalkyloxycarbonyl, aminocarbonyl, alkylaminocarbonyl, dialkylaminocarbonyl; sulfurated derivatives such as alkylthio, arylthio, alkylsulfonyl, arylsulfonyl, alkylsulfinyl, arylsulfinyl, arylsulfonyloxy, aminosulfonyl, alkylaminosulfonyl or dialkylaminosulfonyl.

16. A compound of formula (I) as defined in claim 9, optionally in the form of a pharmaceutically acceptable salt, selected from the group consisting of:
1. 3-amino-1-ethoxycarbonyl-5-[(2-methylthio)pyrimidin-4-yl]-4,6-dihydropyrrolo[3,4-c]pyrazole,
2. 1-ethoxycarbonyl-3-(4-fluoro-benzamido)-5-[(2-methylthio)pyrimidin-4-yl]-4,6-dihydropyrrolo[3,4-c]pyrazole,
3. 3-(4-tert-butyl-benzamido)-1-ethoxycarbonyl-5-[(2-methylthio)pyrimidin-4-yl]-4,6-dihydropyrrolo[3,4-c]pyrazole,
4. 1-ethoxycarbonyl-3-(3-phenoxy-benzamido)-5-[(2-methylthio)pyrimidin-4-yl]-4,6-dihydropyrrolo[3,4-c]pyrazole,
5. 3-(4-fluoro-benzamido)-5-[(2-propylamino)pyrimidin-4-yl]-4,6-dihydropyrrolo[3,4-c]pyrazole,
6. 3-(4-tert-butyl-benzamido)-5-[(2-propylamino)pyrimidin-4-yl]-4,6-dihydropyrrolo[3,4-c]pyrazole,
7. 3-(3-phenoxy-benzamido)-5-[(2-propylamino)pyrimidin-4-yl]-4,6-dihydropyrrolo[3,4-c]pyrazole,
8. 3-(4-fluoro-benzamido)-5-[(2-benzylamino)pyrimidin-4-yl]-4,6-dihydropyrrolo[3,4-c]pyrazole,
9. 3-(4-tert-butyl-benzamido)-5-[(2-benzylamino)pyrimidin-4-yl]-4,6-dihydropyrrolo[3,4-c]pyrazole,
10. 3-(3-phenoxy-benzamido)-5-[(2-benzylamino)pyrimidin-4-yl]-4,6-dihydropyrrolo[3,4-c]pyrazole,
11. 3-(4 fluoro-benzamido)-5-[2-(N-morpholino)pyrimidin-4-yl]-4,6-dihydropyrrolo[3,4-c]pyrazole,
12. 3-(4-tert-butyl-benzamido)-5-[2-(N-morpholino)pyrimidin-4-yl]-4,6-dihydropyrrolo[3,4-c]pyrazole,
13. 3-(3-Phenoxy-benzamido)-5-[2-(N-morpholino)pyrimidin-4-yl]-4,6-dihydropyrrolo[3,4-c]pyrazole,
14. 3-(4-fluoro-benzamido)-5-[2<methyl-piperazino)pyrimidin-4-yl]-4,6-dihydropyrrolo[3,4-c]pyrazole,
15. 3-(4-tert-butyl-benzamido)-5-[2-(4-methyl-piperazino)pyrimidinyl-4-yl]-4,6-dihydropyrrolo[3,4-c]pyrazole,
16. 3-(3-phenoxy-benzamido)-5-[2-(4-methyl-piperazino)pyrimidin-4-yl]-4,6-dihydropyrrolo[3,4-c]pyrazole,
17. 3-(4-fluoro-benzamido)-5-[2-(4-methyl-piperidino)pyrimidin-4-yl]-4,6-dihydropyrrolo[3,4-c]pyrazole,
18. 3-(4-tert-butyl-benzamido)-5-[2-(4-methyl-piperidino)pyrimidin-4-yl]-4,6-dihydropyrrolo[3,4-c]pyrazole,
19. 3-(3-phenoxy-benzamido)-5-[2-(4-methyl-piperidino)pyrimidin-4-yl]-4,6-dihydropyrrolo[3,4-c]pyrazole,
20. 3-(4-fluoro-benzamido)-5-[2-(4-dimethyl-ethylendiamino)pyrimidin-4-yl]-4,6-dihydropyrrolo[3,4-c]pyrazole,
21. 3-(4-tert-butyl-benzamido)-5-[2-(4-dimethyl-ethylendiamino)pyrimidin-4-yl]-4,6-dihydropyrrolo[3,4-c]pyrazole,
22. 3-(3-phenoxy-benzamido)-5-[2-(4-dimethyl-ethylendiamino)pyrimidin-4-yl]-4,6-dihydropyrrolo[3,4-e]pyrazole,
23. 3-(4-fluoro-benzamido)-5-[2-(1,4,4-trimethyl-ethylendiamino)pyrimidin-4-yl]-4,6-dihydropyrrolo[3,4-c]pyrazole,
24. 3-(4-tert-butyl-benzamido)-5-[2-(1,4,4-trimethyl-ethylendiamino)pyrimidin-4-yl]-4,6-dihydropyrrolo[3,4-c]pyrazole,
25. 3-(3-phenoxy-benzamido)-5-[2-(1,4,4-trimethyl-ethylendiamino)pyrimidin-4-yl]-4,6-dihydropyrrolo[3,4-c]pyrazole,
26. 3-(4-fluoro-benzamido)-5-[2-(4-hydroxymethyl-piperidino)pyrimidin-4-yl]-4,6-dihydropyrrolo[3,4-c]pyrazole,
27. 3-(4-tert-butyl-benzamido)-5-[2-(4-hydroxymethyl-piperidino)pyrimidin-4-yl]-4,6-dihydropyrrolo[3,4-c]pyrazole,
28. 3-(3-phenoxy-benzamido)-5-[2-(4-hydroxymethyl-piperdino)pyrimidin-4-yl]-4,6-dihydropyrrolo[3,4-c]pyrazole,
29. 3-(4-fluoro-benzamido)-5-{2-[4-(2-hydroxyethyl)piperazino]pyrimidin-4-yl}-4,6-dihydropyrrolo[3,4-c]pyrazole,
30. 3-(4-tert-butyl-benzamido)-5-{2-[4-(2-hydroxyethyl)piperazino]pyrimidin-4-yl}-4,6-dihydropyrrolo[3,4-c]pyrazole,
31. 3-(3-phenoxy-benzamido)-5-{2-[4-(2-hydroxyethyl)piperazino]pyrimidin-4-yl}-4,6-dihydropyrrolo[3,4-c]pyrazole,
32. 3-(4-fluoro-benzamido)-5-[(2-cyclopropylamino)pyrimidin-4-yl]-4,6-dihydropyrrolo[3,4-c]pyrazole,
33. 3-(4-tert-butyl-benzamido)-5-[(2-cyclopropylamino)pyrimidin-4-yl]-4,6-dihydropyrrolo[3,4-c]pyrazole,
34. 3-(3-phenoxy-benzamido)-5-[(2-cyclopropylamino)pyrimidin-4-yl]-4,6-dihydropyrrolo[3,4-c]pyrazole,
35. 3-(4-tert-butyl-benzamido)-5-[(2-phenylamino)pyrimidin-4-yl]-4,6-dihydropyrrolo[3,4-c]pyrazole,
36. 3-(4-fluoro-benzamido)-5-[(2-phenylamino)pyrimidin-4-yl]-4,6-dihydropyrrolo[3,4-c]pyrazole,
37. 3-(3-phenoxy-benzamido-5-[(2-phenylamino)pyrimidin-4-yl]-4,6-dihydropyrrolo[3,4-c]pyrazole,
38. 3-(4-fluoro-benzamido)-5-[(2-ethyloxy)pyrimidin-4-yl]-4,6-dihydropyrrolo[3,4-c]pyrazole,
39. 3-(4-tert-butyl-benzamido)-5-[(2-ethyloxy)pyrimidin-4-yl]-4,6-dihydropyrrolo[3,4-c]pyrazole,
40. 3-(3-phenoxy-benzamido)-5-[(2-ethyloxy)pyrimidin-4-yl]-4,6-dihydropyrrolo[3,4-c]pyrazole,
41. 3-(4-fluoro-benzamido)-5-[2-(1-methylpiperidin-4-yloxy)pyrimidin-4-yl]-4,6-dihydropyrrolo[3,4-c]pyrazole,
42. 3-(4-tert-butyl-benzamido)-5-[2-(1-methylpiperidin-4-yloxy)pyrimidin-4-yl]-4,6-dihydropyrrolo[3,4-c]pyrazole,
43. 3-(3-phenoxy-benzamido)-5-[2-(1-methylpiperidin-4-yloxy)pyrimidin-4-yl]-4,6-dihydropyrrolo[3,4-c]pyrazole,
44. 1-ethoxycarbonyl-3-(2-naphtalenacetamido)-5-[(2-methylthio)pyrimidin-4-yl]-4,6-dihydropyrrolo[3,4-c]pyrazole,
45. 3-(4-fluoro-benzamido)-5-[6chloro-(pyrimidin-4-yl)]-4,6-dihydropyrrolo[3,4-c]pyrazole,
46. 3-(4-fluoro-benzamido)-5-(thiazol-2-yl)-4,6-dihydropyrrolo[3,4-c]pyrazole,
47. 3-(4-fluoro-benzamido)-5-(benzothiazol-2-yl)-4,6-dihydropyrrolo[3,4-c]pyrazole,
48. 3-(4-tert-butyl-benzamido)-5-[pyrimidin-2-yl]-4,6-dihydropyrrolo[3,4-c]pyrazole,
49. 3-(2-naphtalenacetamido)-5-[pyrimidin-2-yl]-4,6-dihydropyrrolo[3,4-c]pyrazole,
50. 3-amino-1-ethoxycarbonyl-5-[(2-methylthio)pyrimidin-4-yl]-4,6-pyrrolo[4,3-c]4,5,6,7-tetrahydropyridine,
51. 1-ethoxycarbonyl-3-(4-fluoro-benzamido)-5-[(2-methylthio)pyrimidin-4-yl]-pyrazolo[4,3-c]4,5,6,7-tetrahydropyridine,
52. 3-(4-tert-butyl-benzamido)-1-ethoxycarbonyl-5-[(2-methylthio)pyrimidin-4-yl]-pyrazolo[4,3-c]4,5,6,7-tetrahydropyridine,
53. 1-ethoxycarbonyl-3-(3-phenoxy-benzamido)-5-[(2-methylthio)pyrimidin-4-yl]-pyrazolo[4,3-c]4,5,6,7-tetrahydropyridine,
54. 3-(4-fluoro-benzamido)-5-[(2-propylamino)pyrimidin-4-yl]-pyrazolo[4,3-c]4,5,6,7-tetrahydropyridine,
55. 3-(4-tert-butyl-benzamido)-5-[(2-propylamino)pyrimidin-4-yl]-pyrazolo[4,3-c]4,5,6,7-tetrahydropyridine,
56. 3-(3-phenoxy-benzamido)-5-[(2-propylamino)pyrimidin-4-yl]-pyrazolo[4,3-c]4,5,6,7-tetrahydropyridine,
57. 3-(4-fluoro-benzamido)-5-[(2-benzylamino)pyrimidin-4-yl]-pyrazolo[4,3-c]4,5,6,7-tetrahydropyridine,
58. 3-(4-tert-butyl-benzamido)-5-[(2-benzylamino)pyrimidin-4-yl]-pyrrolo[4,3-c]4,5,6,7-tetrahydropyridine,
59. 3-(3-phenoxy-benzamido)-5-[(2-benzylamino)pyrimidin-4-yl]-pyrazolo[4,3-c]4,5,6,7-tetrahydropyridine,
60. 3-(4-fluoro-benzamido)-5-[(2-N-morpholino)pyrimidin-4-yl]-pyrazolo[4,3-c]4,5,6,7-tetrahydropyridine,
61. 3-(4-tert-butyl-benzamido)-5-[(2-N-morpholino)pyrimidin-4-yl]-pyrazolo[4,3-c]4,5,6,7-tetrahydropyridine,
62. 3-(3-phenoxy-benzamido)-5-[(2-N-morpholino)pyrimidin-4-yl]-pyrazolo[4,3-c]4,5,6,7-tetrahydropyridine.

17. A compound of formula (I) as defined in claim 9, optionally in the form of a pharmaceutically acceptable salt, selected from those reported in the experimental section.

18. A process for preparing the compounds of formula (I) and the pharmaceutically acceptable salts thereof, as defined in claim 9, which process comprises:
a) reacting under acidic or basic conditions a compound of formula (II) 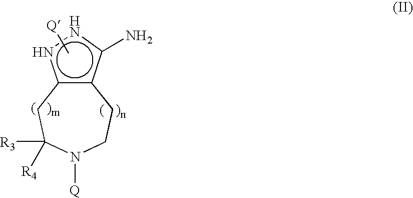
wherein Q represents a suitable nitrogen protecting group and Q′ represents R₂ or a suitable nitrogen protecting group and wherein R₂, R₃, R₄, m and n are as defined in claim 9; so as to obtain a compound of formula (III) 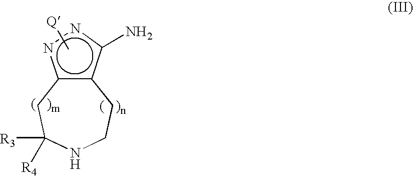
b) reacting the compound of formula (III) with a derivative of formula (IV)
X—R₁ (IV)
wherein R₁ is as defined in claim 9 and X represents a halogen atom or a suitable leaving group, so as to obtain a compound of formula (V) 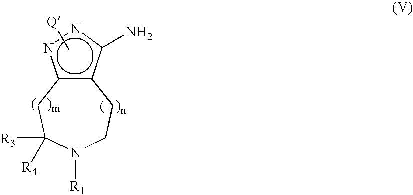
c) reacting the compound of formula (V) with a suitable derivative of formula (VI), (VII), (VIII), (IX), (X) or (XI)
R′COX (VI);
R′OCOX (VII);
R′NCO (VIII);
H₂N—C(═NH)NH₂ (IX);
XSO₂R′ (X);
XSO₂NR′R″ (XI)
wherein R′ and R″ are as defined in claim 9 and X is a halogen atom or suitable leaving group, so as to obtain the corresponding compound of formula (I) below 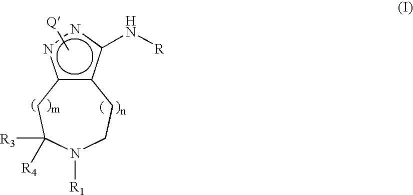
and, optionally,
d) converting it into another compound of formula (I) or into a pharmaceutically acceptable salt thereof.

19. The process of claim 18 wherein, within the compound of formula (II), Q is tert-butoxycarbonyl and Q′ is a hydrogen atom or a group R₂ of formula —COOR′ wherein R′ is ethyl.

20. The process of claim 18 wherein the compound of formula (II) is treated under acidic conditions in the presence of trifluoroacetic or hydrochloric acid.

21. The process of claim 18 wherein X is a chlorine atom or a suitable leaving group selected from alkylsulfonyl or arylsulfonyl.

22. A process for preparing the compounds of formula (I) and the pharmaceutically acceptable salts thereof, as defined in claim 9, which process comprises:
e) hydrolyzing under acidic or basic conditions the compound of formula (I) being obtained in step (c) of claim 18 and wherein R, R₁, R₃, R₄, m and n have the above reported meanings and Q′ is a suitable pyrazole nitrogen protecting group 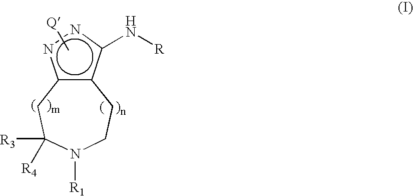
and reacting the thus obtained compound bearing a hydrogen atom in place of Q′ in the presence of a suitable polymeric resin (P), so as to obtain the resin supported compound of formula (XII) 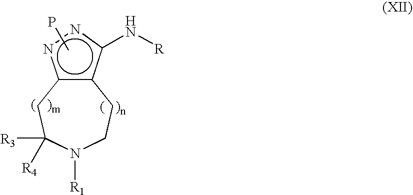
f) optionally converting the compound of formula (XII) into another compound of formula (XII), and
g) cleaving the polymeric resin so as to obtain the desired compound of formula (I) and, whenever desired, converting it into a pharmaceutically acceptable salt thereof.

23. The process of claim 22 wherein, in step (e), the polymeric resin (P) is 2-chloro-trityl chloride resin, trityl chloride resin, p-nitrophenyl carbonate Wang resin or isocyanate polystirenic resin.

24. A library of two or more pyrazole derivatives represented by formula (I) 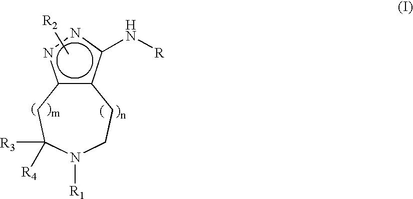
wherein
R is a hydrogen atom or a group selected from —COR′, —COOR′, —CONHR′, —C(═NH)NHR′, —SO₂R′ or —SO₂NR′R″;
R₁ is an optionally substituted 5 or 6 membered heterocyclic group with from 1 to 3 heteroatoms or heteroatomic groups selected from N, NR′, O or S, optionally benzocondensed;
R₂ is hydrogen or it is selected from the group consisting of R′, —COR′, —COOR′, —CONR′R″ or —S(O)_{q}R′;
R₃ and R₄ are both hydrogen atoms or methyl groups or, together with the carbon atom to which they are attached, form a cyclopropyl group;
R′ and R″ are, the same or different and independently in each of the above occasions, a hydrogen atom or an optionally substituted group selected from straight or branched C₁-C₆ alkyl, C₃-C₆ cycloalkyl, aryl, aryl C₁-C₆ alkyl, heterocyclyl or heterocyclyl C₁-C₆ alkyl;
m and n are 0 or 1, provided that they are not both 1;
q is 0 or an integer from 1 to 2;
and the pharmaceutically acceptable salts thereof.

25. A pharmaceutical composition comprising an effective amount of a pyrazole of formula (I) as defined in claim 9 and, at least, one pharmaceutically acceptable excipient, carrier or diluent.

26. A pharmaceutical composition according to claim 25 further comprising one or more chemotherapeutic agents, as a combined preparation for simultaneous, separate or sequential use in anticancer therapy.

27. A product or kit comprising a compound of formula (I) as defined in claim 9, or a pharmaceutical composition thereof as defined in claim 25, and one or more chemotherapeutic agents, as a combined preparation for simultaneous, separate or sequential use in anticancer therapy.

28. A compound of formula (I) or a pharmaceutically acceptable salt thereof, as defined in claim 9, for use as a medicament

29. Use of a compound of formula (I) or a pharmaceutically acceptable salt thereof, as defined in claim 9, in the manufacture of a medicament for treating diseases caused by and/or associated with an altered protein kinase activity.

30. Use according to claim 29 for the treatment of tumors.
